(19) United States
(12) Patent Application Publication
(10) Pub. No.: US 2024/0009256 A1
(43) Pub. Date: Jan. 11, 2024

(54) USE OF PROBIOTIC BACTERIUM-DERIVED EXTRACELLULAR MICRO- AND/OR NANOPARTICLES FOR THE TREATMENT OF DISEASE

(71) Applicant: University of Louisville Research Foundation, Inc., Louisville, KY (US)

(72) Inventors: Wenke Feng, Prospect, KY (US); Craig J. McClain, Louisville, KY (US)

(73) Assignee: University of Louisville Research Foundation, Inc., Louisville, KY (US)

(21) Appl. No.: 17/797,219

(22) PCT Filed: Feb. 4, 2021
(86) PCT No.: PCT/US2021/016626

§ 371 Date: Aug. 3, 2022

Related U.S. Application Data
(60) Provisional application No. 62/969,902, filed on Feb. 4, 2020.

(51) Int. Cl. A61K 35/747; A61K 9/127; A61K 31/405; A61K 31/404; A61P 1/16; A61P 1/04; C12N 1/20

(52) U.S. Cl. CPC A61K 35/747; A61K 9/127; A61K 31/405; A61K 31/404; A61P 1/16; A61P 1/04; C12N 1/20

(57) ABSTRACT

Provided are probiotic bacterium-derived extracellular micro- and/or nanoparticles. Also provided are methods for purifying the probiotic bacterium-derived extracellular micro- and/or nanoparticles and methods for using the disclosed probiotic bacterium-derived extracellular micro- and/or nanoparticles to treat liver diseases and/or disorders including but not limited to acute liver failure (ALT), alcoholic liver disease (ALD), non-alcoholic liver disease, alcoholic hepatitis (AH), liver steatosis, liver fibrosis and/or cholestatic liver disease; increasing intestinal aryl hydrocarbon receptor (AhR) activity, Nrf2 signaling, IL-22 expression, regenerating islet-derived 3β (Reg3P) expression, and/or regenerating islet-derived 3γ (Reg3y) expression; maintaining gut microbiota homeostasis; preventing or reducing bacterial intestinal transcytosis; increasing intestinal tight junctions; decreasing circulating LPS concentration; protecting intestinal barrier integrity against oxidative stress; regulating intestinal Nrf2 signaling; increasing intestinal EGF secretion, hepatic macrophage HB-EGF cleavage and activation; and hepatic EGER activation.

### GOVERNMENT INTEREST

This invention was made with government support under grant numbers GM113226, ES023716-5120, AA024337, AA023190, AA023681, AA022489, AA026926, AA026934, and AA026980 awarded by the National Institutes of Health. The government has certain rights in the invention.

### TECHNICAL FIELD

The presently disclosed subject matter relates generally to compositions and methods for treating diseases and/or disorders. In some embodiments, the compositions comprise bacterium-derived microvesicles and/or nanovesicles/nanoparticles.

### BACKGROUND

Live bacteria need to colonize the intestine to maintain their activity under various luminal conditions. Disease conditions vary from patient to patient, due to the augmentation of pathogenic bacteria. It is therefore unclear whether probiotic treatment can result in sustained changes to the composition of the microbiota. In addition, medications used by patients may be harmful to probiotics. This causes a variable effect of probiotic treatment with live bacteria. Moreover, the clinically recommended dose of probiotics usually consists of billions of live bacteria. Generally, probiotics are considered safe, but several reports have raised safety concerns about ingesting such large numbers of bacteria, especially when the intestinal function and the patient's immune response are compromised.

Using probiotic fermentation supernatant for the prevention/treatment of disease in animal models has been demonstrated in many studies. Usually, this supernatant is gavaged to animals. However, consuming a large volume of supernatant is inconvenient for test animals as well as for patients. Furthermore, the supernatant contains a large numbers of components, and it is difficult to identify individual beneficial compounds.

Exosomes are small microvesicles that are released from cell bodies. Exosomes contain protein, lipid, miRNAs, mRNA, and other metabolites that can be transferred to recipient cells with enhanced cargo delivery compared to other lipid vesicles. Probiotic bacteria have been demonstrated to be effective in disease prevention and treatment. However, how probiotic bacteria exert their effects is largely unknown.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to probiotic bacterium-derived microvesicle compositions and methods of using the same for the treatment of disease. Composition are provided that comprise a macrovesicle derived from a strain of probiotic bacteria.

The presently disclosed subject matter also relates in some embodiments to the methods for treating disease. In some embodiments, a method for treating disease is provided that comprises administering to a subject in need thereof an effective amount of a probiotic bacterium-derived microvesicle composition as described herein. In some embodiments, the disease is selected from the group consisting of sepsis, acute liver failure, alcoholic liver disease, non-alcoholic liver disease, liver fibrosis and inflammatory bowel disease.

For administration of a probiotic bacterium-derived microvesicle composition described herein, in some embodiments, the composition is administered orally to thereby treat the disease. In some embodiments, administering the composition reduces an amount of pro-inflammatory cytokine in subject, including, in some embodiments, a reduction in an amount of interleukin 1β, tumor necrosis factor-1α, interleukin-6. In some embodiments, administering the composition reduces liver fat content, liver cell death, and serum ALT and AST activities.

More particularly, in some embodiments the presently disclosed subject matter relates to probiotic bacterium-derived extracellular microparticles and/or nanoparticles (NPs). In some embodiments, the probiotic bacterium is Lactobacillus rhamnosus GG (LGG). In some embodiments, the probiotic bacterium-derived extracellular micro- and/or nanoparticle is isolated from culture supernatant in which the probiotic bacterium is growing. In some embodiments, the probiotic bacterium-derived extracellular micro- and/or nanoparticle is purified from the culture supernatant to a purity of at least 50%, 60%, 70%, 75%, 80%, 85%, 90%, 95%, 96%, 97%, 98%, or 99% with respect to other components of the culture supernatant.

In some embodiments, a probiotic bacterium-derived extracellular micro- and/or nanoparticle as disclosed herein encapsulates or is otherwise associated with a tryptophan catabolic metabolite, optionally indoleacrylic acid (IA), indole-3-aldehyde (I3A), 3-methyleneoxindole, indole, indole-3-lactic acid (ILA), indole acetic acid (IAA), or any combination thereof.

In some embodiments, the presently disclosed subject matter also relates to methods for isolating probiotic bacterium-derived extracellular micro- and/or nanoparticles. In some embodiments, the methods comprise growing probiotic bacterium in culture, recovering some or all of the culture medium in which the probiotic bacterium is growing, and isolating the probiotic bacterium-derived extracellular micro- and/or nanoparticle from the culture medium.

In some embodiments, the presently disclosed subject matter also relates to methods for increasing probiotic LGG growth and LGG-derived extracellular micro- and/or nanoparticles. In some embodiments, the methods comprise using amino acids and/or small molecules in LGG cultural medium in which probiotic bacterium is growing faster and/or produces enhanced amount of extracellular micro- and/or nanoparticle and/or bacterium-derived AhR ligands. In some embodiments, the isolating procedure comprises use of a sucrose gradient and ultracentrifugation to separate the probiotic bacterium-derived extracellular micro- and/or nanoparticle from other components of the culture medium.

In some embodiments, the presently disclosed subject matter also relates to methods for treating liver diseases and/or disorders. In some embodiments, the methods comprise administering to subjects in need thereof effective amounts of the probiotic bacterium-derived extracellular micro- and/or nanoparticles disclosed herein to ameliorate at least one symptom of the liver disease or disorder. In some embodiments, the liver disease and/or disorder is selected from the group consisting of acute liver failure (ALF), alcoholic liver disease (ALD), non-alcoholic liver disease, liver steatosis, liver fibrosis, cholestatic liver disease or any combination thereof.

In some embodiments, the presently disclosed subject matter also relates to methods for increasing intestinal aryl hydrocarbon receptor (AhR) activity, Nrf2 signaling, IL-22 expression, regenerating islet-derived 3β (Reg3β) expression, regenerating islet-derived 3γ (Reg3γ) expression, or any combination thereof. In some embodiments, the methods comprise administering to a cell, tissue or organ, optionally a cell, tissue, or organ present within a subject, a probiotic bacterium-derived extracellular micro- and/or nanoparticle as disclosed herein in an amount and via a route sufficient to increase intestinal aryl hydrocarbon receptor (AhR) activity, Nrf2 signaling, IL-22 expression, Reg3β expression, Reg3γ expression, or any combination thereof in the cell, tissue or organ.

In some embodiments, the presently disclosed subject matter also relates to methods for maintaining gut microbiota homeostasis, preventing or reducing bacterial intestinal transcytosis, or any combination thereof. In some embodiments, the methods comprise administering to a cell, tissue or organ, optionally a cell, tissue, or organ present within a subject, a probiotic bacterium-derived extracellular micro- and/or nanoparticle as disclosed herein in an amount and via a route sufficient to maintain gut microbiota homeostasis and/or prevent and/or reduce bacterial intestinal transcytosis.

In some embodiments, the presently disclosed subject matter also relates to methods for increasing intestinal tight junctions. In some embodiments, the methods comprise administering to a cell, tissue or organ, optionally a cell, tissue, or organ present within a subject, a probiotic bacterium-derived extracellular micro- and/or nanoparticle as disclosed herein in an amount and via a route sufficient to increase intestinal tight junctions.

In some embodiments, the presently disclosed subject matter also relates to methods for decreasing circulating LPS concentration. In some embodiments, the methods comprise administering to a cell, tissue or organ, optionally a cell, tissue, or organ present within a subject, a probiotic bacterium-derived extracellular micro- and/or nanoparticle as disclosed herein in an amount and via a route sufficient to decrease circulating LPS concentration.

In some embodiments, the presently disclosed subject matter also relates to methods for protecting intestinal barrier integrity against oxidative stress, optionally oxidative stress induced by alcohol. In some embodiments, the methods comprise administering to a cell, tissue or organ, optionally a cell, tissue, or organ present within a subject, a probiotic bacterium-derived extracellular micro- and/or nanoparticle as disclosed herein in an amount and via a route sufficient to protect intestinal barrier integrity against oxidative stress.

In some embodiments, the presently disclosed subject matter also relates to methods for increasing intestinal EGF secretion, In some embodiments, the methods comprise administrating to a cell tissue or organ, optionally a cell, tissue, or organ present within a subject, a probiotic bacterium-derived extracellular micro- and/or nanoparticle of as disclosed herein in an amount and via a route sufficient to increase intestinal EGF secretion.

In some embodiments, the presently disclosed subject matter also relates to methods for increasing HB-EGF activation, the methods comprising administrating to a cell tissue or organ, optionally a cell, tissue, or organ present within a subject, a probiotic bacterium-derived extracellular micro- and/or nanoparticle as disclosed herein in an amount and via a route sufficient to increase macrophage HB-EGF cleavage and activation. In some embodiments, the administering is associated with upregulation of intestinal Nrf2 signaling.

Thus, it is an object of the presently disclosed subject matter to provide compositions and methods for treating diseases and/or disorders.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1F. Characterization of LGG-derived exosome-like nanoparticles (LDNPs).

FIG. 1A. Representative TEM image of LDNPs and the frequency of observed nanoparticles (NPs) by diameter. FIG. 1B. Size (left panel) and protein concentration (right panel) comparison between LDNPs and MRS-derived NPs (MRS-NPs). FIG. 1C. Coomassie blue staining of protein bands on SDS-polyacrylamide gel (SDS-PAGE). FIG. 1D. Western-blot for CD63 protein in LDNPs and Caco-2-derived NPs. FIG. 1E. Up-take of PKH67-labeled LDNPs in the ileum, mesenteric adipose tissue (MAT), and liver tissue. LDNPs were labeled with PKH67, and orally gavaged to mice at 10 μg/g. 12 hours later, tissues were collected and florescence was recorded. The images show that the majority of LDNPs are taken up by intestinal tissue and MAT. FIG. 1F. Up-take of LDNPs in macrophages RAW264.7 and hepatocytes Hepa1-6. Raw264.7 and Hepa1-6 cells were incubated with PKH67-labeled LDNPs (0.2 μg/ml) for 6 hours. After washing, florescence was recorded. The images show that the macrophages are able to take up large amounts of LDNPs, whereas hepatocytes are unable to taken up large amount of LDNPs. Arrows indicating PKH67 positive staining of LDNPs. DAPI 463 was used for nucleic counter staining.

FIGS. 2A-2F. LDNPs inhibited LPS-induced inflammation in macrophages. RAW264.7 cells FIGS. 2A-2D: Dose-dependent effects of LDNPs on relative Tnfα mRNA expression with or without LPS stimulation (FIG. 2A, left panel); relative mRNA expression of inflammatory mediators (FIG. 2A, right panel) and protein levels of TNF-α and IL-10 (FIG. 2B) after LDNPs and LPS treatment. FIG. 2C. The effects of LDNPs-depletion in LGGs on LPS-induced Tnfα mRNA expression. FIG. 2D. The effects of LDNPs on Tnfα and Il1b mRNA expression is time-dependent. LDNPs inhibited LPS-induced Tnfα and Il1b mRNA expression in peritoneal macrophages (FIG. 2E) and bone marrow-derived macrophages (BMDM) (FIG. 2F). Data shown represent the Mean±SEM of at least 3 independent experiments performed in triplicate. *p<0.05, **p<0.01, ***p<0.001, ****p<0.0001.

FIGS. 3A and 3B: LDNPs treatment prevented LPS-induced sepsis. Female C57BL/6 mice were treated with LDNPs (5 mg/kg) by I.P. injection and, 22 hours later, the mice were I.P. injected LPS (5 mg/kg). The mice were sacrificed (depicted in top panel of FIG. 3A). FIG. 3A is a series of bar graphs of serum levels of pro-inflammatory cytokine TNF-α and IL-1β. FIG. 3B is a plot of a comparison of survival rates between Control and LPS groups with or without LDNPs pretreatment. Female C57BL/6 mice were divided into 4 groups, mice were pretreated with LDNPs 5 mg/kg for 24 hours and LPS 10 mg/kg by I.P. injection. The imaging show that the LDNP treatment improves the survival rates of the mice. *p<0.05; **p<0.01; ***p<0.001.

FIGS. 4A and 4B: LDNPs inhibited LPS-induced pro-inflammatory cytokine expression in peritoneal macrophage and bone marrow derived macrophage. Macrophages were incubated with LDNPs (0.2 μg protein/ml) for 20 hours, after which LPS (100 ng/ml) was added to the culture for 4 hours (depicted in the top panel of the FIG. 4A). TNFα and IL-β mRNA levels were analyzed in peritoneal macrophages (PM; FIG. 4A) and bone marrow-derived macrophages (BMDM; FIG. 4B). *p<0.05; **p<0.01; ***p<0.001.

FIGS. 5A-5C: LDNPs prevented LPS/GalN-induced acute liver failure. Female C57BL/6 mice were treated with LDNPs (5 mg/kg) via I.P. injection for 26 hours and at the last 6 hours LPS (50 μg/kg) and GalN (300 mg/kg) were injected via IP (Depicted in the tope panel of the FIG. 5A). FIG. 5A is a series of bar graphs showing the levels of ALT and AST in serum. Data are expressed as Mean±SEM. *p<0.05, **p<0.01, ***p<0.001. FIG. 5B is a series of photographs showing gross morphology of the livers, showing that LDNP treatment preserved liver overall morphology. FIG. 5C is a series of representative images of H&E staining of liver sections, also showing that the liver architecture was well preserved in LDNPs pretreatment group.

FIGS. 6A and 6B: LDNPs protected against LPS/GalN-induced hepatic apoptosis. FIG. 6A is a series of representative images of TUNEL staining of liver. FIG. 6B is a photograph of relative levels of proteins related to apoptosis. determined by immunoblot.

FIGS. 7A-7D: LDNPs protected against LPS/GalN-induced inflammation. FIG. 7A is a bar graph of serum TNF-α, protein levels determined by ELISA. FIGS. 7B-7D are bar graphs of relative mRNA levels of the inflammatory cytokines Tnfα (FIG. 7B) and IL6 (FIG. 7C), and Tlr4 (FIG. 7D) in liver tissues. Data are expressed as Mean±SEM. *p<0.05, **p<0.01, ***p<0.001.

FIGS. 8A-8E: LDNPs protected against LPS/GalN-induced inflammasome activation. FIG. 8A is a bar graph of IL-10 protein levels in serum determined by ELISA. FIG. 8B is an immunoblot of relative protein levels. FIGS. 8C-8E are bar graphs of the mRNA expression of Nlrp3 (FIG. 8C), Caspase 1 (FIG. 8D), and IL1β (FIG. 8E) levels of inflammasome activation in the liver, respectively. Data are expressed as Mean±SEM. *p<0.05, **p<0.01, ***p<0.001.

FIGS. 9A and 9B: LDNPs enhance hepatic EGFR phosphorylation. FIG. 9A is a representative western blot of p-EGFR, Total-EGFR, PI3K, p-Akt, total-Akt, and β-actin protein levels in liver. FIG. 9B is a series of bar graphs of relative mRNA expression levels of EGFR ligands Egf and Hb-egf. Mice were treated as described previously. Data are expressed as Mean±SEM. *p<0.05, **p<0.01, ***p<0.001.

FIGS. 10A and 10B: LDNPs increased HB-EGF paracrine function in macrophages and hepatocytes. Raw264.7 cells were treated with LDNPs (0.2 μg/ml) for 24 hours (depicted in the top panel of the FIG. 10A). The bottom panel of FIG. 10A is a pair of bar graphs of relative mRNA levels of EGFR ligands HB-Egf and Egf measured by RT-qPCR. FIG. 10B is an immunoblot of relative P-Egfr Tyr1068 protein levels in Hepa1-6 cells treated with LDNP-conditioned medium (RAW264.7 cells) for 2 hours or LDNPs for 24 hours (depicted in the top panel of the FIG. 10B). β-actin is included as a loading control. Data are expressed as Mean±SEM. *p<0.05, **p<0.01.

FIG. 11: LDNPs increased paracrine function in peritoneal and bone marrow derived macrophages, and hepatocytes. Peritoneal macrophages (PM) and bone marrow derived macrophages (BMDM) were treated with LDNPs 0.2 μg/ml for 24 hours and the conditioned medium was used to treat AML-12 cells for 2 hours (depicted in the top panel of the FIG. 11). Relative P-Egfr Tyr1068 and Total Egfr protein levels were determined by immunoblot. β-actin is included as a loading control.

FIG. 12: LDNPs increased macrophage metalloprotease activity. Raw264.7 cells, PMs, BMDM and AML-12 cells were treated with LDNPs at 0.2 μg/ml for 24 hours. Activation of MMP-9 (92 kDa) and MMP-2 (72 kDa) were assayed for proteinases by zymographic analysis.

FIGS. 13A and 13B: LDNPs-mediated activation of EGFR in macrophages. Raw264.7 cells were pretreated with LDNPs (0.2 μg/ml) or the EGFR inhibitor AG1478 (150 nM) for 20 hours and then treated with LPS for 4 hours to induce inflammation response (depicted in the top panel of the FIG. 13A). The bottom panel of FIG. 13A is a representative western blot of p-EGFR, Total-EGFR, PI3K, p-Akt, total-Akt, and β-actin. FIG. 13B is a bar graph of relative mRNA levels of pro-inflammatory cytokines Tnf-α and Il-1β. Data are expressed as Mean±SEM. *p<0.05, **p<0.01, ***p<0.001.

FIGS. 14A-14D: LDNPs stimulated the secretion of EGF in the duodenal Brunner's gland. FIG. 14A is a bar graph of duodenal mRNA expression levels of Egf in C57BL/6 mice treated with LDNPs (5 mg/kg) by oral gavage for various duration times. FIG. 14B is a bar graph of relative duodenum and ileum mRNA levels in C57BL/6 mice treated with LDNPs (5 mg/kg) by oral gavage for 12 hours (depicted in the top panel of the FIG. 14B). FIG. 14C is a bar graph of serum levels of EGF protein. FIG. 14D is an immunoblot of relative protein levels in liver tissues. Data are expressed as Mean±SEM. *p<0.05, **p<0.01.

FIGS. 15A and 15B: Effect of LDNPs treatment on duodenal EGF secretion. Female C57BL/6 mice were treated with LDNPs (5 mg/kg) via oral gavage for 12 hours, and the duodenum tissues were harvested for organ culture (Depicted in the top panel of the FIG. 15A). FIG. 15A is a bar graph of EGF protein levels in duodenum organ culture medium. FIG. 15B is a series of immunoblots showing that LDNPs-conditioned duodenal secretions increased hepatocyte EGFR signaling AML-12, Hepa1-6, and Caco-2 cells (Depicted in the top panel of the FIG. 15B). Duo-CM: Duodenum Conditioned Medium. Data are expressed in Mean±SEM, **p<0.01.

FIGS. 16A-16C: LDNPs improved alcohol-induced steatosis. C56BL/6 mice were subjected to the NIAAA (10d+1b) alcohol model. At day 7, LDNPs were gavaged once a day for 3 days (Depicted in the top panel of the FIG. 16A). FIGS. 16A and 16B are series of representative images of H&E and oil red O staining of liver sections, respectively. FIG. 16C is a bar graph of hepatic levels of various triglycerides in treated mice. Data are expressed in Mean±SEM (N=7). **p<0.01; ***p<0.001.

FIGS. 17A and 17B: LDNPs improved alcohol-induced liver injury and hepatocyte apoptosis. C56BL/6 mice were subjected to NIAAA (10d+1b) alcohol model. At day 7, LDNPs were gavaged once a day for 3 days as described in FIG. 16. FIG. 17A is a bar graph of serum ALT and AST levels. FIG. 17B is a series of representative images of TUNEL staining of liver sections. Data are expressed in Mean±SEM (N=7). **p<0.01; ***p<0.001.

FIGS. 18A-18E. LDNPs increased AhR reporter activity and intestinal downstream signaling. FIG. 18A. AhR reporter activity of MRS, LDNPs, LGGs, and LDNP-depleted LGGs (LGGs(np-d)). FIG. 18B. Signal intensity of IA and I3A. The standard curve study by LC-MS showed a linear representation in the signal range shown in Y-axes. FIG. 18C. Upper panel: the effects of the AhR inhibitor, CH229131, on LDNPs-induced upregulation of Cyp1a1 and Il22 mRNA expression in lamina propria lymphocytes (LPLs); lower panel: IL-22 protein level in the culture medium of LPLs. AhR ligand I3A: positive control. FIG. 18D. Relative mRNA expression of Il22 and Cyp2E1 in mouse ileum and colon. FIG. 18E. Relative 483 mRNA expression of Reg3γ and Reg3β in mouse ileum and colon. Data shown as Mean 484±SEM, n=5. *p<0.05, **p<0.01, ***p<0.001.

FIGS. 19A-19F. LDNPs increased intestinal tight junction expression in Caco-2 cells. FIG. 19A. Western blot for ZO-1, Occludin, and Claudin-1 protein in Caco-2 cell lysates. FIG. 19B. Relative Cyp1a1 mRNA expression (upper panel) and Cyp1a1 activity (lower panel) in Caco-2 cells treated with LDNPs, CH223191 (AhR inhibitor), ML385 (Nrf2 inhibitor) and I3A. FIG. 19C. Western blot for tight junction proteins in Caco-2 cell lysates. FIG. 19D. Western blot for Nrf2 protein in cell lysates of LDNPs-treated Caco-2 cells. FIG. 19E. Relative mRNA expression of Nrf-2 in Caco-2 cells treated with LDNPs, CH223191, ML385 and I3A. I3A was used as an AhR ligand control. FIG. 19F. The effects of CH223191 or ML385 on Nrf2 protein level in Caco-2 cells treated with LDNPs. Data shown represent the Mean±SEM of at least 3 independent experiments performed in triplicate for cell culture studies. *p<0.05, **p<0.01, ***p<0.001.

FIGS. 20A-20F. LDNPs reversed/prevented alcohol-associated liver disease. FIG. 20A. Experimental design of animal treatment. FIG. 20B. Representative microphotographs of H&E (upper panel) and Oil red O (lower panel) stained mouse liver sections. FIG. 20C. Hepatic triglyceride levels. FIG. 20D. Serum ALT and AST levels. FIG. 20E. Representative microphotographs of TUNEL-stained mouse liver sections. FIG. 52F. Hepatic Tnfα and Il1b mRNA expression. Data are expressed in Mean±SEM (n=5-7 mice/group). PF: pair-fed; AF: alcohol-fed. *p<0.05, **p<0.01, ***p<0.001.

FIGS. 21A-21F. LDNPs increased intestinal AhR activity and decrease hepatic bacterial translocation. Relative ileum Cyp1a1 mRNA expression (FIG. 21A) and activity (FIG. 21B) in the tissue of LDNPs-treated PF- or AF-fed mice. FIG. 21C. Relative ileum Il-22 mRNA expression (upper panel) and serum IL-22 protein level (lower panel). FIG. 21D. The effects of LDNPs on ileum mRNA expression of Reg3b and Reg3g. FIG. 21E. Upper panel: representative microphotographs of immunofluorescence staining for lysozyme on mouse ileum tissue. Lower panel: quantification of lysozyme-positive stained Paneth cells (red). DAPI (blue): nucleic counter stain. FIG. 21F. Fold-change of bacteria load in the livers of LDNPs-treated PF- or AF-fed mice. Data are expressed in Mean±SEM (n=5-7 mice/group). *p<0.05, **p<0.01, ***p<0.001.

FIGS. 22A-22F. LDNP treatment decreased circulating endotoxin level through Nrf2 activation. FIG. 22A. Western blot for nuclear Nrf2 protein in ileum tissues of LDNPs-treated mice fed with alcohol. Histone H3 serves as a loading control. FIGS. 22B and 22C. Relative mRNA level of Nrf2 and Nqo1 in mouse ileum. FIG. 22D. DHE staining for the measurement of ROS in the ileum tissues. FIG. 22E. Western blots for tight junction proteins in the ileum tissue. FIG. 22F. Serum endotoxin levels. Data are expressed in Mean±SEM (n=5-7 mice/group). *p<0.05.

FIGS. 23A-23E. The effects of LDNPs in ALD are regulated by AhR signaling pathway. FIG. 23A. Upper panel: H & E staining of liver tissues (left) and hepatic triglyceride levels (right) of alcohol-fed mice that co-administered with LDNPs and control vehicle (CV) or AhR inhibitor CH223191; Lower panel: serum ALT and AST. FIG. 23B. Serum IL-22 protein levels (left panel); relative ileum mRNA expression of Il22 and Cyp1a1 (middle and right panel). FIG. 23C. Ileum Reg3g mRNA expression. FIG. 23D. Fold-change of hepatic bacterial load. FIG. 23E. Ileum mRNA expression of Nrf2 and Nqo1. Data are expressed in Mean±SEM (n=5-7 mice/group). FIG. 23F. Proposed model of LDNP action on intestinal AhR signaling in ALD. *p<0.05, **p<0.01, ***p<0.001.

FIG. 24. Coomassie blue staining of SDS-PAGE gel of LDNPs incubated in pH 2.2 solution at 37° C. for 2 hours.

FIG. 25. Alcohol and LDNPs treatment did not change hepatic Il-22 and Cyp1a1 mRNA expression. Mice were treated as described in the Materials and Methods for the EXAMPLES section below.

### DETAILED DESCRIPTION

Alcohol-associated liver disease (ALD) is a major cause of mortality. Gut barrier dysfunction-induced bacterial translocation and endotoxin release contribute to the pathogenesis of ALD. Probiotic Lactobacillus rhamnosus GG (LGG) is known to be beneficial on experimental ALD through reinforcing the intestinal barrier function.

Probiotics have been used to prevent/treat a variety of digestive diseases including ALD. Live probiotics need to colonize the gut to exert their function. Unfortunately, underlying disease states provide an unfavorable environment for probiotic bacterial gut colonization, which diminishes probiotics' function. In last few years, we showed that LGG culture supernatant (LGGs, without live bacteria) was effective in the prevention of ALD in experimental models of acute and chronic alcohol exposure in mice. However, how LGG supernatant exerts its therapeutic effects is not fully understood.

Recent studies show that bacteria, both Gram-negative and Gram-positive, produce NPs. The NPs derived from “bad” bacteria were demonstrated to be pathogenic. However, “good” bacteria-, probiotics-derived NPs have not been studied. As disclosed herein, administration of LGG-derived exosome-like NPs (LDNPs) effectively reversed ALD in the binge-on-chronic alcohol exposure mouse model, suggesting that probiotic LGGs may exert its function through LDNPs in ALD. Administration of LDNPs markedly increased intestinal aryl hydrocarbon receptor (AhR) activity, IL-22 and regenerating islet-derived 3 (Reg3β and Reg3γ) expression, which play a key role in maintaining gut microbiota homeostasis and preventing bacterial intestinal transcytosis. In addition, LDNPs administration significantly increased intestinal tight junctions and decreased circulating LPS concentration, associated with upregulation of intestinal Nrf2 signaling, which is known for protecting intestinal barrier integrity against oxidative stress induced by alcohol. Metabolomic analysis revealed that LDNPs contain high levels of AhR ligands, which are microbial metabolites of tryptophan. The presently disclosed subject matter is thus consistent with LDNPs increasing intestinal Reg3 expression by activating intestinal AhR-Nrf2 signaling, thereby modulating gut microbiota homeostasis and enhance intestinal barrier function, leading to the suppression of ALD.

As disclosed herein, whether the beneficial effects of probiotic bacteria could result from delivering probiotic bacterium-derived molecules to recipient host cells via exosomes was tested. Extracellular nanoparticles from probiotic bacterium Lactobacillus rhamnosus GG (LGG) cultural supernatant (referred to here as “LDNPs” or “LGG-derived nanoparticles”) was isolated and tested. LDNPs were used to treat acute liver failure and alcoholic liver disease in murine models. LDNPs were effective in reducing lipopolysaccharide/D-galactosamine (LPS/GalN)-induced liver cell death and in reducing alcohol-induced liver injury.

Particularly, LDNPs increased tight junction protein expression in epithelial cells and protected from the lipopolysaccharide (LPS)-induced inflammatory response in macrophages. Three-day oral application of LDNPs protected the intestine from alcohol-induced barrier dysfunction and the liver from steatosis and injury in an animal model of ALD. Co-administration of an aryl hydrocarbon receptor (AhR) inhibitor abolished the protective effects of LDNPs, indicating that the effects are mediated, at least in part, by intestinal AhR signaling.

Furthermore, LDNP administration increased intestinal IL-22-Reg3 and nuclear factor erythroid 2-related factor 2 (Nrf2)-tight junction signaling pathways leading to the inhibition of bacterial translocation and endotoxin release in ALD mice. This protective effect was associated with LDNP enrichment of bacterial tryptophan metabolites that are AhR agonists.

It thus appears that probiotic bacteria secrete microvesicles that contain functional compounds and signaling materials, which provide at least a fraction of the probiotic action. Isolation of these microvesicles could be used to enrich the beneficial signaling ingredients while simultaneously removing both harmful and non-useful components.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims.

The term “about”, as used herein to refer to a measurable value such as an amount of weight, time, etc., is meant to encompass in some embodiments variations of ±20%, in some embodiments ±10%, in some embodiments ±5%, in some embodiments ±1%, in some embodiments ±0.1%, in some embodiments ±0.5%, and in some embodiments ±0.01% from the specified amount, as such variations are appropriate to perform the disclosed methods.

As used herein, the term “and/or” when used in the context of a list of entities, refers to the entities being present singly or in any possible combination or subcombination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a method of the presently disclosed subject matter can “consist essentially of” one or more enumerated steps as set forth herein, which means that the one or more enumerated steps produce most or substantially all of the intended result to be produced by the claimed method. It is noted, however, that additional steps can be encompassed within the scope of such a method, provided that the additional steps do not substantially contribute to the result for which the method is intended.

With respect to the terms “comprising”, “consisting essentially of”, and “consisting of”, where one of these three terms is used herein, the presently disclosed and claimed subject matter can include the use of either of the other two terms. Similarly, it is also understood that in some embodiments the methods of the presently disclosed subject matter comprise the steps that are disclosed herein, in some embodiments the methods of the presently disclosed subject matter consist essentially of the steps that are disclosed, and in some embodiments the methods of the presently disclosed subject matter consist of the steps that are disclosed herein.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

Pharmaceutical compositions comprising the present compositions are administered to an individual in need thereof by any number of routes including, but not limited to, topical, oral, rectally, vaginally, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the bacteria of the presently disclosed subject matter. More particularly, such compounds can be formulated as pharmaceutical compositions using standard pharmaceutically acceptable carriers, fillers, solubilizing agents and stabilizers known to those skilled in the art.

The presently disclosed subject matter also encompasses the use pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically-acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the presently disclosed subject matter may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container.

In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the presently disclosed subject matter formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the presently disclosed subject matter.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Remington's Pharmaceutical Sciences. 18th ed. (1990) Mack Publishing, Easton, Pennsylvania, United States of America, which is incorporated herein by reference.

Typically, dosages of the composition of the presently disclosed subject matter which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In one embodiment, the dosage of the compound will vary from about 10 μg to about 10 g per kilogram of body weight of the animal. In another embodiment, the dosage will vary from about 10 mg to about 1 g per kilogram of body weight of the subject.

The composition may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the sex and age of the subject, etc.

### II. Compositions of the Presently Disclosed Subject Matter

Bacterial extracellular nanoparticles and ALD. Exosomes are a type of extracellular nanoparticles (NPs) that are produced in most cell types and carry a variety of genetic materials (miRNA, mRNA, and other noncoding RNAs), proteins and metabolites. Recent studies suggest that exosomes function as natural effectors of signaling between cells and across various tissues through the transfer of their cargos. In the past, it was thought that only Gram-negative bacteria produced NPs, but we now know that Gram-positive bacteria can also release NPs during their growth. Although further studies are needed to thoroughly understand the mechanisms underlying the biogenesis of bacterial NPs, use of bacterial NP as biomarkers and pharmabiotics is a rapidly emerging field. Recent studies showed that probiotic derived NPs can inhibit liver cancer cell growth and enhance immune response to infection. Our preliminary data showed that LGG-derived exosome-like NPs (LDNPs) reversed ALD in mice. These preliminary studies suggest that the beneficial effects of LGG, and more relevantly, LGGs, are likely mediated by LDNPs.

In some embodiments, the presently disclosed subject matter relates to extracellular micro- and/or nanoparticles that have been isolated from bacteria, which in some embodiments can be bacteria (alternatively referred to herein as “probiotics”). Such extracellular micro- and/or nanoparticles are referred to herein as a “bacterium-derived”, in some embodiments “probiotic bacterium-derived”, extracellular micro- and/or nanoparticle. As used herein, the phrase “extracellular micro- and/or nanoparticles that have been isolated from bacteria” refers to extracellular particles that can be isolated from the culture media of bacteria growing in culture, which in some embodiments are nanoscale particles. In some embodiments, the bacterium from which the extracellular particle is isolated is a probiotic bacterium, which in some embodiments can be Lactobacillus rhamnosus GG (LGG).

As disclosed herein, in some embodiments the bacterium-derived extracellular micro- and/or nanoparticle (optionally a probiotic bacterium-derived extracellular micro- and/or nanoparticle) is isolated from culture supernatant in which the bacterium (optionally the probiotic bacterium) is growing.

In some embodiments, the bacterium-derived extracellular micro- and/or nanoparticle is purified from the culture supernatant to a purity of at least 50%, 60%, 70%, 75%, 80%, 85%, 90%, 95%, 96%, 97%, 98%, or 99% with respect to other components of the culture supernatant. In some embodiments, purification of the bacterium-derived extracellular micro- and/or nanoparticle is performed via centrifugation, including but not limited to the method disclosed herein (see Materials and Methods of the EXAMPLES, LGG culture and LDNP isolation). Other methods of purifying extracellular micro- and/or nanoparticles can also be employed, including but not limiting to immunoprecipitation or any other methodology that relies on binding of an antibody or a fragment or derivative thereof to any antigen that is present on or in the extracellular micro- and/or nanoparticle.

As such, in some embodiments the presently disclosed subject matter relates to method for isolating a probiotic bacterium-derived extracellular micro- and/or nanoparticle, the method comprising growing probiotic bacterium in culture, recovering some or all of the culture medium in which the probiotic bacterium is growing, and isolating the probiotic bacterium-derived extracellular micro- and/or nanoparticle from the culture medium.

In some embodiments, the probiotic bacterium is an IGG bacterium. Additionally it can be beneficial to increase the growth of LGG in culture in order to maximize recovery of extracellular micro- and/or nanoparticles from the culture medium in which the LGG is growing. As such, in some embodiments the presently disclosed subject matter relates to methods for increasing probiotic LGG growth and LGG-derived extracellular micro- and/or nanoparticle recovery, the method comprising using amino acids and/or small molecules in LGG cultural medium in which probiotic bacterium is growing faster and/or produces enhanced amount of extracellular micro- and/or nanoparticle and/or bacterium-derived AhR ligands. In some embodiments, the isolating procedure comprises use of a sucrose gradient and ultracentrifugation to separate the probiotic bacterium-derived extracellular micro- and/or nanoparticle from other components of the culture medium.

The extracellular micro- and/or nanoparticles of the presently disclosed subject matter can act as carriers for active agents, as desired. Thus, in some embodiments the extracellular micro- and/or nanoparticle of the presently disclosed subject matter can encapsulate or otherwise be associated with a tryptophan catabolic metabolite, optionally indoleacrylic acid (IA), indole-3-aldehyde (I3A), 3-methyleneoxindole, indole, indole-3-lactic acid (ILA), indole acetic acid (IAA), or any combination thereof.

### III. Uses and Methods of Use of the Presently Disclosed Compositions

Gut microbiota, probiotics and alcoholic liver disease (ALD). Despite extensive research into mitigating its effects, alcohol remains one of the most common causes of both acute and chronic liver disease in the United States. Importantly, there is no FDA-approved therapy for any stage of ALD. Recent studies have laid a solid foundation on the role of gut microbiota on ALD development and progression. Alcohol consumption causes gut dysbiosis with the overgrowth of “harmful” bacteria and reduced “beneficial” bacteria. Strategies targeting to restore the eubiosis have received increased attention in the prevention/treatment of ALD.

As disclosed herein. Lactobacillus rhamnosus GG (LGG), one of the best-characterized probiotic strains, reversed established ALD in mice. However, the application of live probiotics in a clinical situation has generated mixed results. Viable probiotics must colonize the gut to exert their function, but disease conditions and the use of medications, in particular, antibiotics, provide an unfavorable environment for probiotic gut colonization. In addition, overgrowth of the probiotics can cause side effects. As set forth herein, however, LGG culture supernatant (LGGs), without viable probiotic bacteria, is effective in preventing experimental ALD.

Intestinal barrier function and ALD. Research in the last decade has clearly demonstrated that intestinal barrier dysfunction is one of the key mechanisms in the development of ALD. The gut barrier consists of a physical barrier as well as immune surveillance. The physical barrier includes the mucus layer and epithelial cells, which prevent bacterial access to host epithelial cell surface and penetration of bacterial products through the epithelial cells, which are connected by junction proteins such as tight junction (TJ) and adherens junction (AJ) proteins. However, it is clear that viable bacteria cannot pass the small intercellular space between enterocytes even with disrupted junction proteins. Gut immune regulation prevents bacterial translocation resulting from increased trans-cytosis. Disrupted mucosal and epithelial junction and immune surveillance function by alcohol allow bacterial product translocation and increase endotoxemia, which activate hepatic Kupffer cells and cause hepatic steatosis and injury. Our preliminary data showed that LDNPs treatment significantly increased intestinal TJ proteins in mice fed alcohol, and in intestinal epithelial Caco-2 cells. We also showed that hepatic bacterial translocation was markedly reduced by LDNPs treatment. These data suggest that LDNPs treatment improves gut barrier function. However, the mechanism(s) underlying the effects are unknown.

Intestinal AhR and Nrf2 pathways in ALD. Intestinal commensal bacteria affect host metabolism and immune regulation through generation of metabolic products, such as tryptophan metabolites. Tryptophan has been the most studied amino acid in relation to alcohol and alcoholism. Supplementation with tryptophan reduced alcohol consumption through serotonin, a host metabolite of tryptophan. Microbial tryptophan metabolism produces indoles and their derivatives, of which many are aryl hydrocarbon receptor (AhR) agonists. AhR, expressed in intestinal type 3 intestinal innate lymphoid cells (ILC3), is a ligand-activated nuclear receptor, and whether AhR activation is beneficial or detrimental is ligand-dependent. While AhR is known for its ability to regulate toxic effects of environmental chemicals, recent studies demonstrate that microbes or natural AhR ligands are beneficial in regulating immune response by producing IL22. Fecal levels of AhR ligands have been shown to be lower in patients with AH, and supplementation of indole-3-acetic acid (IAA), a microbial tryptophan metabolite and an AhR ligand, protected mice against ALD by increasing intestinal IL22 and C-type lectin, regenerating islet-derived 3γ (Reg3γ), which play a critical role in maintaining gut microbiota homeostasis and inhibiting bacterial translocation. Interestingly, our preliminary studies identified several abundant AhR ligands, such as indole-3-acrylic acid (IA), indole-3-aldehyde (I3A) and IAA, in LGGs. Strikingly, when the LDNPs were removed, the concentrations of IA and I3A in the residual supernatant were markedly reduced. Moreover, LGGs and LDNPs, but not NPs-depleted LGGs (LGGs(npd)), increased AhR reporter activity, indicating that those AhR microbial agonists are mainly packed in the LDNPs. In addition, we showed that LDNPs treatment increased intestinal Il22 and Reg3 (β and γ) mRNA expression in a mouse model of ALD. These results strongly suggest that LDNPs activate AhR. It is thus likely that the LDNPs-associated decrease in alcohol-induced bacterial translocation is mediated by intestinal AhR-IL22-Reg3 signaling. On the other hand, alcohol metabolism-induced oxidative stress and inflammation causes disruption of intestinal intercellular connections by decreasing TJ proteins. We showed that LDNPs treatment increased nuclear factor erythroid 2-related factor 2 (Nrf2) protein, a master regulator of cellular defense mechanisms against oxidative stress, in Caco-2 cells and in intestinal tissues of mice fed alcohol, along with increased expression of TJ proteins, ZO-1, occludin and claudin-1, and decreased serum endotoxin concentration. Whether the effects of LDNPs on AhR and Nrf2 signaling are interrelated is unknown, but crosstalk between AhR and Nrf2 pathways has been suggested. These preliminary studies thus suggest that LDNPs may suppress alcohol-induced increased gut permeability through an AhR-Nrf2 pathway.

Enhancement of the effects of LGG. As described above, using LDNPs is likely superior to viable LGG because there is no need for bacterial colonization and growth in the gut and is superior to LGGs due to the enrichment of beneficial metabolites. To this end, in vitro manipulation of LGG to enhance the LDNPs biogenesis and to enrich the beneficial components of LGGs is a plausible strategy to improve LDNP function.

Thus, in some embodiments the presently disclosed subject matter relates to methods for treating a liver disease or disorder, the method comprising administering to a subject in need thereof an effective amount of the composition comprising a bacterium-derived extracellular micro- and/or nanoparticle as disclosed herein to ameliorate at least one symptom of the liver disease or disorder. In some embodiments, the liver disease or disorder is selected from the group consisting of acute liver failure (ALF), alcoholic liver disease (ALD), non-alcoholic liver disease, liver steatosis, liver fibrosis, cholestatic liver disease or any combination thereof.

Intestinal barrier function and ALD. Research in the last decade has clearly demonstrated that intestinal barrier dysfunction is one of the key mechanisms in the development of ALD. The gut barrier consists of a physical barrier as well as immune surveillance. The physical barrier includes the mucus layer and epithelial cells, which prevent bacterial access to host epithelial cell surface and penetration of bacterial products through the epithelial cells, which are connected by junction proteins such as tight junction (TJ) and adherens junction (AJ) proteins. However, it is clear that viable bacteria cannot pass the small intercellular space between enterocytes even with disrupted junction proteins. Gut immune regulation prevents bacterial translocation resulting from increased trans-cytosis. Disrupted mucosal and epithelial junction and immune surveillance function by alcohol allow bacterial product translocation and increase endotoxemia, which activate hepatic Kupffer cells and cause hepatic steatosis and injury. Our preliminary data showed that LDNPs treatment significantly increased intestinal TJ proteins in mice fed alcohol, and in intestinal epithelial Caco-2 cells. We also showed that hepatic bacterial translocation was markedly reduced by LDNPs treatment. These data suggest that LDNPs treatment improves gut barrier function. However, the mechanism(s) underlying the effects are unknown.

Intestinal AhR and Nrf2 pathways in ALD. Intestinal commensal bacteria affect host metabolism and immune regulation through generation of metabolic products, such as tryptophan metabolites. Tryptophan has been the most studied amino acid in relation to alcohol and alcoholism. Supplementation with tryptophan reduced alcohol consumption through serotonin, a host metabolite of tryptophan. Microbial tryptophan metabolism produces indoles and their derivatives, of which many are aryl hydrocarbon receptor (AhR) agonists. AhR, expressed in intestinal type 3 intestinal innate lymphoid cells (ILC3), is a ligand-activated nuclear receptor, and whether AhR activation is beneficial or detrimental is ligand-dependent. While AhR is known for its ability to regulate toxic effects of environmental chemicals, recent studies demonstrate that microbes or natural AhR ligands are beneficial in regulating immune response by producing IL22. Fecal levels of AhR ligands have been shown to be lower in patients with AH, and supplementation of indole-3-acetic acid (IAA), a microbial tryptophan metabolite and an AhR ligand, protected mice against ALD by increasing intestinal IL22 and C-type lectin, regenerating islet-derived 3γ (Reg3γ), which play a critical role in maintaining gut microbiota homeostasis and inhibiting bacterial translocation. Interestingly, our preliminary studies identified several abundant AhR ligands, such as indole-3-acrylic acid (IA), indole-3-aldehyde (I3A) and IAA, in LGGs. Strikingly, when the LDNPs were removed, the concentrations of IA and I3A in the residual supernatant were markedly reduced. Moreover, LGGs and LDNPs, but not NPs-depleted LGGs (LGGs(npd)), increased AhR reporter activity, indicating that those AhR microbial agonists are mainly packed in the LDNPs. In addition, we showed that LDNPs treatment increased intestinal Il22 and Reg3 (β and γ) mRNA expression in a mouse model of ALD. These results strongly suggest that LDNPs activate AhR. It is thus likely that the LDNPs-associated decrease in alcohol-induced bacterial translocation is mediated by intestinal AhR-IL22-Reg3 signaling. On the other hand, alcohol metabolism-induced oxidative stress and inflammation causes disruption of intestinal intercellular connections by decreasing TJ proteins. We showed that LDNPs treatment increased nuclear factor erythroid 2-related factor 2 (Nrf2) protein, a master regulator of cellular defense mechanisms against oxidative stress, in Caco-2 cells and in intestinal tissues of mice fed alcohol, along with increased expression of TJ proteins, ZO-1, occludin and claudin-1, and decreased serum endotoxin concentration. Whether the effects of LDNPs on AhR and Nrf2 signaling are interrelated is unknown, but crosstalk between AhR and Nrf2 pathways has been suggested. These preliminary studies thus suggest that LDNPs may suppress alcohol-induced increased gut permeability through an AhR-Nrf2 pathway.

Thus, in some embodiments the presently disclosed subject matter relates to methods for increasing intestinal aryl hydrocarbon receptor (AhR) activity, Nrf2 signaling, IL-22 expression, regenerating islet-derived protein 3β (Reg3b) expression, regenerating islet-derived protein 3γ (Reg3g) expression, or any combination thereof. In some embodiments, the methods comprise, consist essentially of, or consist of administering to a cell, tissue or organ, optionally a cell, tissue, or organ present within a subject, an effective amount of a composition comprising, consisting essentially of, or consisting of a bacterium-derived extracellular micro- and/or nanoparticle of the presently disclosed subject matter in an amount and via a route sufficient to increase intestinal aryl hydrocarbon receptor (AhR) activity, Nrf2 signaling, IL-22 expression, Reg3β expression, Reg3γ expression, or any combination thereof in the cell, tissue or organ.

Enhancement of the effects of LGG. As described above, using LDNPs is likely superior to viable LGG because there is no need for bacterial colonization and growth in the gut and is superior to LGGs due to the enrichment of beneficial metabolites. To this end, in vitro manipulation of LGG to enhance the LDNPs biogenesis and to enrich the beneficial components of LGGs is a plausible strategy to improve LDNP function.

Maintaining gut microbiota homeostasis. In some embodiments, the presently disclosed subject matter also relates to methods for maintaining gut microbiota homeostasis, preventing or reducing bacterial intestinal transcytosis, or any combination thereof. In some embodiments, the methods comprise, consist essentially of, or consist of administering to a cell, tissue or organ, optionally a cell, tissue, or organ present within a subject, an effective amount of a composition comprising, consisting essentially of, or consisting of a bacterium-derived extracellular micro- and/or nanoparticle of the presently disclosed subject matter in an amount and via a route sufficient to maintain gut microbiota homeostasis and/or prevent and/or reduce bacterial intestinal transcytosis.

Applications to tight junction integrity. As disclosed herein, the bacterium-derived extracellular micro- and/or nanoparticle of the presently disclosed subject matter and compositions comprising the same can be employed for maintaining or enhancing various aspects of tight junction biology including but not limited to the number of tight junctions present (for example, in the intestine) and/or the structural integrity thereof. Thus, in some embodiments the presently disclosed subject matter relates to methods for increasing intestinal tight junctions comprising, consisting essentially of, or consisting of administering to a cell, tissue or organ, optionally a cell, tissue, or organ present within a subject, an effective amount of a composition comprising, consisting essentially of, or consisting of a bacterium-derived extracellular micro- and/or nanoparticle of the presently disclosed subject matter in an amount and via a route sufficient to increase intestinal tight junctions.

Similarly, in some embodiments the presently disclosed subject matter relates to methods for protecting intestinal barrier integrity against oxidative stress, optionally oxidative stress induced by alcohol. Thus, in some embodiments the presently disclosed subject matter relates to methods comprising, consisting essentially of, or consisting of administering to a cell, tissue or organ, optionally a cell, tissue, or organ present within a subject, an effective amount of a composition comprising, consisting essentially of, or consisting of a bacterium-derived extracellular micro- and/or nanoparticle of the presently disclosed subject matter in an amount and via a route sufficient to protect intestinal barrier integrity against oxidative stress.

Applications related to EGF secretion and activation. As disclosed herein LDNPs have been shown to modulate epidermal growth factor and its biological activities. As such, in some embodiments the presently disclosed subject matter relates to methods for increasing intestinal EGF secretion that comprise, consist essentially of, or consist of administrating to a cell tissue or organ, optionally a cell, tissue, or organ present within a subject, further optionally an intestinal cell or a tissue comprising the same an effective amount of a composition comprising, consisting essentially of, or consisting of a bacterium-derived extracellular micro- and/or nanoparticle of the presently disclosed subject matter in an amount and via a route sufficient to increase intestinal EGF secretion.

Similarly, in some embodiments the presently disclosed subject matter relates to methods for increasing HB-EGF activation. In some embodiments, the presently disclosed methods comprise, consist essentially of, or consist of administrating to a cell tissue or organ, optionally a cell, tissue, or organ present within a subject, an effective amount of a composition comprising, consisting essentially of, or consisting of a bacterium-derived extracellular micro- and/or nanoparticle of the presently disclosed subject matter in an amount and via a route sufficient to increase macrophage HB-EGF cleavage and activation.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

LGG culture and LDNP isolation. LGG was purchased from American Type Culture Collection (ATCC 53103, Rockville, Maryland, United States of America) and cultured in autoclaved deMan, Rogosa & Sharpe (MRS) broth at 37° C. for 40 hours. The culture density was measured with a spectrophotometer at OD600. The culture suspension (2×10⁹ CFU/ml) was centrifuged at 2,000 g for 10 minutes, at 5,000 g for 20 minutes, and then at 10,000 g for 30 minutes to eliminate debris including dead cells and other waste materials. The obtained supernatant was filtered and ultracentrifuged at 150,000 g for 70 minutes (Optima L-100XP Ultra Centrifuge, Beckman Coulter, Atlanta, Georgia, United States of America). After ultracentrifugation, the supernatants were collected and stored (nanoparticles-depleted LGGs, or LGGs (np-d)), and the pellet containing LDNPs was washed in phosphate-buffered saline (PBS), ultracentrifuged, resuspended in PBS, and stored at −80° C. for later use.

Animals and treatments. C57BL/6J mice (6-8 weeks of age) from Jackson Laboratory (Bar Harbor, Maine, United States of America) were maintained at 22° C. with a 12 hour light/dark cycle and initially had free access to a normal chow diet and tap water. Mice were then fed the Lieber DeCarli Diet containing 5% alcohol (w/v) (Alcohol-fed, AF) or isocaloric maltose dextrin (Pair-fed, PF). For the AF groups, mice were initially fed the control Lieber-DeCarli liquid diet (Bio-Serve, Flemington, New Jersey, United States of America) for 5 days to acclimate to the liquid diet. The content of alcohol in the liquid diet was gradually increased from 1.6% (w/v) to 5% (w/v) in the next 6 days and remained at 5% for the subsequent 10 days. Mice in PF group were fed isocaloric maltose dextrin in substitution for alcohol in the liquid diet. On experimental Day 10, a bolus of EtOH (5 g/kg body weight) was given to AF mice by gavage 9 hours before harvesting, while mice in PF groups received a gavage of isocaloric maltose dextrin (10d+1b model). LDNPs were administered to mice in the last 3 days by daily gavage of 200 μL of LDNPs (50 μg protein content). AhR inhibitor, CH223191 (Sigma-Aldrich, St. Louis, Missouri, United States of America) was gavaged at 10 mmol/kg in last three days. Control mice were gavaged with an equal volume of control vehicle (PBS).

Statistics. Statistical analyses were performed using the statistical computer package, GraphPad Prism version 6, (GraphPad Software Inc., San Diego, California, United States of America). Results are expressed as means± standard error of the mean (SEM). Statistical comparisons were made using two-way analysis of variance (ANOVA) with Tukey's post hoc test or Student's t-test, where appropriate. Differences were considered to be significant at p<0.05. Significance is noted as *p<0.05, **p<0.01, ***p<0.001 between groups.

Imaging of NPs by transmission electron microscopy (TEM). LDNPs were fixed in 3% glutaraldehyde overnight for protein cross-linking. The glutaraldehyde was then removed by pipetting and the samples were fixed in 3% cacodylate buffered glutaraldehyde (pH 7.3) for three hours. Samples were subsequently post-fixed in cacodylate buffered 1% osmium tetroxide, followed by dehydration through a series of graded alcohols, and then embedded in LX-112 epoxy resin. Sections were cut at 80-100 nm, mounted on 200 mesh copper grids, stained with uranyl acetate and lead citrate, and viewed in a Philips CM12 transmission electron microscope operating at 80 KV. Digital images were acquired with a SIA-7C side mounted CCD digital camera. The diameter frequency was calculated.

Coomassie brilliant blue staining. SDS-PAGE gels were fixed in 25% IPA and 10% HoAC in water for 30-60 minutes. Gels were then stained in 10% acetic acid in water, containing 60 mg/L of Coomassie Blue R-250 Dye (ThermoFisher, Waltham, Massachusetts, United States of America). Bands appeared within 30 minutes and the staining proceeded until desired band intensity is reached. Gels were then destained in 10% acetic acid for 2 hours or more. Images of the gels were then captured and the gels stored in 7% HoAC.

Immunofluorescence. Cryosections were cut at 10 μm thickness and then fixed in acetone:methanol (1:1) at −20° C. for 2 minutes and rehydrated in phosphate buffered saline (PBS) (137 mM sodium chloride, 2.7 mM potassium chloride, 10 mM disodium hydrogen phosphate, and 1.8 mM potassium dihydrogen phosphate). Sections were permeabilized with 0.2% Triton X-100 in PBS for 10 minutes and blocked in 4% nonfat milk in Triton-Tris buffer (150 mM sodium chloride containing 10% Tween-20 and 20 mM Tris, pH 7.4), and then incubated for 24 hours with the primary antibody Lysozyme (Abcam, Cambridge, United Kingdom) followed by secondary antibody incubation for 1 hour (AlexaFluor-594-conjugated antirabbit IgG, ThermoFisher, Massachusetts, United States of America). DAPI was used for nucleic counterstain (Invitrogen Corporation, Carlsbad, California, United States of America). Slides were mounted with PROLONG® Gold brand antifade mountant (Invitrogen) for imaging and quantification of Lysozyme-positive cells.

PKH67 labeling of LDNPs. LDNPs were labeled with florescent dye PKH67 included in the PKH67 Fluorescent Cell Linker Kits (Sigma, St. Louis, Missouri, United States of America) according to the manufacture's instruction. Labeled LDNPs preparation was then gavaged to mice (10 μg/g) and the tissues were collected 12 hours later for analysis. Macrophages RAW264.7 and hepatocytes Hepa1-6 were incubated with PKH67-labled LDNPs (0.2 μg/ml) for 6 hours. The deposition of LDNPs in the tissues and cells was evaluated under a fluorescence microscope by counting the number of PKH67-positive stained cells.

Real-time quantitative PCR Total mRNA was extracted from mouse liver and intestine tissues using Trizol reagent (Life Technologies, Carlsbad, California, United States of America) according to the manufacturer's instruction and reverse-transcribed using cDNA Supermix (QuantaBio, Beverly, Massachusetts, United States of America). Primers used for gene expression analysis were listed in Table 1. 18S and Gapdh were used as internal controls. Real-time qPCR was performed on an ABI 7300 fast real-time PCR thermocycler, where SYBR green PCR Master Mix (Applied Biosystems, Foster City, California, United States of America) was used. The relative gene expression was determined by the ΔΔCT method.

TABLE 1
Primer Sequences for PCR

| | Forward Primer | Reverse Primer |
|---|---|---|
| Gene | (SEQ ID NO:) | (SEQ ID NO:) |
| Tnfα | CCAGCCGATGGGTTGTACCT | TGACGGCAGAGAGGAGGTTG |
| | (SEQ ID NO: 1) | (SEQ ID NO: 2) |
| Il1b | TTCATCTTTGAAGAAGAGCC | TCGGAGCCTGTAGTGCAGTT |
| | CAT | (SEQ ID NO: 4) |
| | (SEQ ID NO: 3) | |
| I16 | TACCACTTCACAAGTCGGAG | CTGCAAGTGCATCATCGTTG |
| | GC | TTC |
| | (SEQ ID NO: 5) | (SEQ ID NO: 6) |
| Mcp1 | CAGCCAGATGCAGTTAACG | TCTCTCTTGAGCTTGGTGAC |
| | (SEQ ID NO: 7) | (SEQ ID NO: 8) |
| Nrf2 | CCAGCTACTCCCAGGTTGC | CCAAACTTGCTCCATGTCCT |
| | (SEQ ID NO: 9) | (SEQ ID NO: 10) |
| Ngo1 | TTTGAGAGAGTGCTCGTAGC | GGTCTTCTTATTCTGGAAAG |
| | (SEQ ID NO: 11) | G |
| | | (SEQ ID NO: 12) |
| Reg3g | ATGCTTCCCCGTATAACCAT | GGCCATATCTGCATCATACC |
| | CA | AG |
| | (SEQ ID NO: 13) | (SEQ ID NO: 14) |
| Reg3b | ACTCCCTGAAGAATATACCC | CGCTATTGAGCACAGATACG |
| | TCC | AG |
| | (SEQ ID NO: 15) | (SEQ ID NO: 16) |
| Cyp1a1 | GACCCTTACAAGTATTTGGT | GGTATCCAGAGCCAGTAACC |
| | CGT | T |
| | (SEQ ID NO: 17) | (SEQ ID NO: 18) |
| I122 | AATCAGCTCAGCTCCTGTCA | TCGCCTTGATCTCTCCACTC |
| | (SEQ ID NO: 19) | (SEQ ID NO: 20) |
| Gapdh | AGGTCGGTGTGAACGGATTT | TGTAGACCATGTAGTTGAGG |
| | G | TCA |
| | (SEQ ID NO: 21) | (SEQ ID NO: 22) |
| 18S | GTAACCCGTTGAACCCCATT | CCATCCAATCGGTAGTAGCG |
| | (SEQ ID NO: 23) | (SEQ ID NO: 24) |

Western Blotting. Protein was extracted from frozen intestine tissues. Western blotting was performed as described in (Shao et al. (2018) Intestinal HIF-1α deletion exacerbates alcoholic liver disease by inducing intestinal dysbiosis and barrier dysfunction. Journal of Hepatology 69:886-895). Antibodies used for western blotting for proteins ZO-1, Occludin, and Claudin-1 were from Cell Signaling (Danvers, Massachusetts, United States of America). Nrf-2, β-actin and Histone H3 antibodies were purchased from Abcam (Cambridge, Massachusetts, United States of America). For western blotting in Caco-2 cell derived NPs and LDNPs, CD 63 antibody from System Bioscience (Palo Alto, California, United States of America) was used.

H&E staining and Terminal deoxynucleotidyl transferase dUTP nick end labeling (TUNEL) assay. For histopathological analysis, H&E staining was performed on paraffin-embedded liver sections. For TUNEL assay staining, formalin-fixed paraffin liver sections were sectioned at 5 μm, and the sections were stained with the ApopTag Peroxidase in situ Apoptosis Detection Kit (Chemicon, California, United States of America) as described in (Zhang et al. (2015) Enhanced AMPK phosphorylation contributes to the beneficial effects of Lactobacillus rhamnosus GG supernatant on chronic-alcohol-induced fatty liver disease. Journal of Nutritional Biochemistry 26:337-344). In brief, the slides were deparaffinized and rehydrated, then treated with proteinase K. Slides were then treated with 3% hydrogen peroxide to quench endogenous peroxidases and incubated with terminal deoxynucleotidyl transferase (TdT) and anti-digoxigenin-peroxidase, respectively. Diaminobenzidine (DAB) was then applied. Hematoxylin was used for nucleic counterstain. Under the microscope, apoptotic cells exhibited a brown nuclear stain and the TUNEL positive were counted.

Cell culture. Mouse macrophages RAW 264.7 and the human intestinal epithelial cells Caco-2 were maintained in DMEM-high glucose and EMEM-high glucose (Corning; 10-009CV) respectively. The medium was supplemented with 10% fetal bovine serum, 1X penicillin-streptomycin solution (100U/ml penicillin, and 100 μg/ml streptomycin; Sigma Aldrich) in a humidified atmosphere (5% CO₂, 95% air, 37° C.). Peritoneal macrophages and bone marrow-derived macrophages (BMDM) were isolated from mice as described in (Ying et al. (2013) Investigation of Macrophage Polarization Using Bone Marrow Derived Macrophages. Journal of Visualized Experiments 76:50323.). Isolated BMDM were then cultured in modified DMEM medium with macrophage colony-stimulating factor (M-CSF), which is a cytokine that directs cell differentiation. Cells were utilized for experimentation at 70-80% confluence. RAW264.7, peritoneal macrophages (PMs) and bone marrow-derived macrophages (BMDMs) were pretreated with LDNPs (0.2 μg/ml for 20 hours) and then treated with E. coli-derived lipopolysaccharides (LPS; O55:B5; Sigma) at 100 ng/mL concentration for 4 hours. Caco-2 cells at 70-80% confluence were treated with LDNPs (0.2 μg/ml), I3A (0.1 mM), the AhR inhibitor, CH-223191 (10 μM) or Nrf2 inhibitor, ML385 (5 μM) for 24 hours. Lamina propria lymphocytes (LPLs) were treated with LDNPs (0.2 μg/ml), I3A (0.1 mM) or the AhR inhibitor, CH223191 (10 μM) for 24 hours.

Isolation of lamina propria lymphocytes (LPLs). Small intestines were harvested and placed in ice-cold Hank's balanced salt solution (HBSS) as described in (Hendrikx et al. (2018) Bacteria engineered to produce IL-22 in intestine induce expression of REG3G to reduce ethanol-induced liver disease in mice. Gut 68:1504-1515). After removal of residual mesenteric fat tissue, Peyer's patches were excised, and small intestines were opened longitudinally. Tissues were washed in ice-cold HBSS and cut into 1 cm pieces. Tissues were then vortexed in 20 mL of HBSS with 5 mM EDTA, 1 mM sodium pyruvate, 25 mM HEPES and 1 mM dithiothreitol at 37° C. at 150 rpm for 20 minutes. The epithelial cell layer was removed by intensive shaking, washed and stored for further use. After washing in 10 mL of HBSS with 5 mM EDTA, 1 mM sodium pyruvate and 25 mM HEPES, small intestine pieces were minced with scissors and digested in serum-free media containing 1 mg/mL Collagenase (Millipore Sigma, Burlington, Massachusetts, United States of America), 0.1 U/mL Dispase (Worthington Biochem, Lakewood, New Jersey, United States of America) and 0.1 mg/mL DNase I (Millipore Sigma) at 37° C. at 150 rpm for 30 minutes. Cells were washed and passed through a 70 mm cell strainer. Cells were resuspended in 4 mL of 40% Percoll and placed in 4 mL of 80% Percoll. Percoll gradient separation was performed by centrifugation for 20 minutes at 600 g at room temperature without brake. Lymphoid fractions were collected at the interphase of the Percoll gradient, washed once and resuspended in culture medium.

AhR-reporter assay. AhR-reporter assay was performed using AhR Reporter Assay system (Indigo Biosciences, Inc., State College, Pennsylvania, United States of America) according to manufacturer's instructions (see also Sing et al. (2019) Enhancement of the gut barrier integrity by a microbial metabolite through the Nrf2 pathway. Nature Communications 10:89). The AhR Reporter cells (expressing luciferase under AhR promoter) as well as positive control MeBio (AhR ligand) compound were provided in the kit.

Intestinal microsomes Cyp1a1 activity. Intestinal microsomes were prepared as described in (Stohs et al. (1976) The isolation of rat intestinal microsomes with stable cytochrome P-450 and their metabolism of benzo(alpha) pyrene. Arch Biochem Biophys 177:105-116). For intestinal microsome preparation, the intestine was removed and washed with ice cold 0.9% sodium chloride to remove luminal contents. The intestine was longitudinally cut open to expose the mucosal layer and the mucosa was scrapped with the help of a glass slide. The scraped tissue was collected in homogenization buffer (50 mM Tris-HCl buffer containing glycerol (20% v/v), protease inhibitor (1%) and heparin (3 U/ml)) to avoid agglutination and degradation of enzyme. This suspended mucosa was homogenized and centrifuged at 10,000×g for 20 minutes at 4° C. Supernatant obtained was further centrifuged at 105,000×g for 60 minutes at 4° C. The pellet was washed with buffer and centrifuged again at 105,000×g for 60 minutes at 4° C. The pellet was suspended in homogenization buffer and used for protein and CYP enzymes assays. The microsomes (20 μg) were used for P450-Glo Cyp1A1 luminescence assays using a luminometer.

Measurement of Cvp1A1 enzyme activity in vitro. Caco-2 cells (50,000 cells/well) were plated in a 48-well plate. Cells were then treated with LDNPs (0.2 μg/ml) or Indole-3-aldehyde (I3A, 0.1 mM) for 24 hours. After treatment, cells were washed to remove any residual LDNPs or I3A, then the wells were replenished with fresh medium containing Cyp1A1 substrate as per the protocol provided with a P450-GLO™ CYP1A1 Assay System brand kit (Promega Corporation, Madison, Wisconsin, United States of America) for 3 hours. After incubation, 25 μl of culture medium was removed from each well and transferred to a 96-well white opaque plate and 25 μl of luciferin detection reagent was added to initiate the luminescence reaction and plate was incubated at room temperature for 20 minutes. After incubation, luminescence was recorded using a luminometer. The data were reported as foldchange over vehicle treatment.

Lipid accumulation. Frozen sections of liver tissue were sliced at 10 μm and stained with Oil-Red-O solution (Sigma, St. Louis, Missouri, United States of America) for 10 minutes, washed, and counterstained with hematoxylin for 45 seconds. Samples were then mounted with CC/Mount (Sigma). Additionally, liver triglyceride (TG) levels were determined using commercial kit according to manufacturer's instructions (Thermo Scientific, Waltham, Massachusetts, United States of America).

ELISA. Protein level of IL-22 in mouse serum and cell culture medium were determined using IL-22 ELISA kit (Thermo Scientific) according to the manufacturer's instructions. Protein levels of IL-1β and TNF-α in cell culture medium were determined using IL-1β and TNF-α ELISA kits (Thermo Scientific).

Endotoxin assay. Chromogenic Limulus amebocyte lysate (LAL) endotoxin kit was used to determine serum LPS levels according to the manufacturer's protocol (Lonza, Basel, Switzerland). The procedure was described in (Shao et al. (2018) Intestinal HIF-1a deletion exacerbates alcoholic liver disease by inducing intestinal dysbiosis and barrier dysfunction. Journal of Hepatology 69:886-895). All materials used for blood sample collecting and endotoxin measurement were pyrogen free.

DHE Staining. Reactive oxygen species (ROS) accumulation in the liver was examined by dihydroethidium (DHE) staining. In brief, cryostat sections of liver were incubated with 5 μmol/L DHE (Molecular Probes, Eugene, Oregon, United States of America) for 30 minutes at 37° C. in the dark. Nonfluorescent dihydroethidium is oxidized by ROS to yield the red fluorescent product, ethidium, which binds to nucleic acids and stains the nucleus with bright fluorescent red. The red fluorescence was examined under confocal microscopy and the intensity of fluorescence was quantified using Image J software.

Metabolomics analysis. Metabolite Extraction from Exosome and Cultural Supernatant. Tryptophan derivatives in cultural supernatant samples were extracted by SPE as described in (He et al. (2019) Simultaneous Quantification of Nucleosides and Nucleotides from Biological Samples. J Am Soc Mass Spectrom 30:987-1000) with some modifications. In brief, 500 μL of the supernatant was loaded onto an OASIS HLB cartridge (Waters Corp., Milford, Massachusetts, United States of America) that had been activated and equilibrated with methanol and water following the manufacturer's instructions. The cartridge was then washed twice by 1 mL diH2O and eluted twice by 100% acetonitrile with 0.01% formic acid. The eluate was combined and lyophilized overnight. For tryptophan derivatives extraction from exosome, 500 μL 50% ethanol was added and vortexed thoroughly. The wall of the exosomes was broken by repeating freeze-thaw for at least 10 times. After centrifugation, the supernatant was uploaded onto an OASIS HLB cartridge. The cartridge was then washed and eluted in the same manner as the culture supernatant. After lyophilizing the eluate, the residue was reconstructed in solvent that has the same content with the starting gradient of LC. The sample was then centrifuged at 14,000 g for 10 min at 4° C. The upper clear solution was transferred to an LC vial for LC-MS analysis.

LC-MS/MS Analysis. A Thermo Q Exactive HF Hybrid Quadrupole-Orbitrap Mass Spectrometer coupled with a Thermo DIONEX UltiMate 3000 HPLC system (Thermo Fisher Scientific) was used. The HPLC system was equipped with an ACQUITY UPLC HSS T3 column (150×2.1 mm i.d., 1.8 μm) purchased from Waters Corp. The temperatures of the column and autosampler were set as 40° C. and 7° C., respectively. The sample injection volume was 2 μL. Mobile phase A was ddH2O with 0.01% formic acid and mobile phase B was pure acetonitrile. The LC gradient was as follows: 0 min, 10% mobile phase B; 0 to 10 min, mobile phase B increased linearly from 10% to 50%; 10 to 14 min, mobile phase B increased linearly from 50% to 100%; and 14 to 18 min, mobile phase B was kept constant at 100%. The flow rate was 0.35 mL/min. The operating parameters for mass spectrometry were the same as in (He et al., 2019), except that the full scan range was changed to 115-300 m/z. All samples were analyzed by LC-MS in random order under negative mode to obtain full MS data for quantification. The group-based pooled samples were analyzed by LC-MS/MS in negative mode to acquire MS/MS spectra at three collision energies (20, 40, and 60 eV) for compound identification. Fourteen tryptophan derivative standards purchased from Sigma-Aldrich Corp. and Cayman Chemical (Ann Arbor, Michigan, United States of America) were also analyzed by LC-MS/MS under negative mode in different collision energies (20, 40, and 60 eV), and the results were recorded in an in-house database.

Data Analysis. XCMS software was used for spectrum deconvolution (8) and MetSign software was used for metabolite identification, cross-sample peak list alignment, and normalization (Wei et al. (2011) A Computational Platform for High-Resolution Mass Spectrometry-Based Metabolomics. Anal Chem. 2011; 83:7668-7675; Wei et al. (2012) Data preprocessing method for liquid chromatography-mass spectrometry based metabolomics. Anal Chem 2012; 84:7963-7971; Wei et al. (2014) Data dependent peak model based spectrum deconvolution for analysis of high resolution LC-MS data. Anal Chem 86:2156-2165). To identify bile acids, the LC-MS/MS data of the pooled samples were matched to the MS/MS spectra of 46 bile acid standards recorded in an in-house database that contained parent ion m/z, MS/MS spectra, and retention time. The threshold for the MS/MS spectrum similarity was set as ≥0.4, and the thresholds of the retention time difference and m/z variation window were set as ≤0.15 min and ≤5 ppm, respectively.

Proteomics analysis. Sample digestion. Samples were analyzed as described in (Teng et al. (2018) Plant-Derived Exosomal MicroRNAs Shape the Gut Microbiota. Cell Host Microbe 24:637-652 e638). Briefly, exosomes isolated from LGG strain ATCC53103 were adjusted to a concentration of 1.65 mg/mL using phosphate-buffered saline. Exosome aliquots (25 μg) were diluted with an equal volume of 2% (w/v) sodium dodecyl sulfate (SDS) in 0.1M Tris-HCl pH8.5 prior and adjusted to 0.1M dithiothreitol (DTT) using a 1M DTT stock solution. The sample was reduced and denatured by heating at 60° C. for 30 minutes in a heating block prior to sample digestion using the filter assisted sample preparation (FASP) protocol in 8M urea. The digested, ultra-filtered samples were trap-cleaned with C18 PROTO™ 300 Å Ultra MicroSpin columns, lyophilized by vacuum centrifugation, and redissolved into 16 μL of 2% v/v acetonitrile and concentrations estimated using absorption at 205 nm by Nanodrop 2000 (Thermo Fisher Scientific, San Jose, California, United States of America) measurement.

LCMS data acquisition. Peptide samples (500 ng) were loaded onto an in house pulled (360 μm OD×100 μm ID) fused silica tip needle tip packed with 12 cm of Aeris Peptide XB-C18 3.6 μm, 100A material (Phenomenex, Torrance, California, United States of America) using a Proxeon EASY n-LC (Thermo-Fisher Scientific) UHPLC system. Peptides were eluted using a 250 nL/min linear gradient of 2% v/v acetonitrile/0.1% v/v formic acid to 40% v/v acetonitrile/0.1% v/v formic acid over 45 minutes. The sample was introduced into an LTQ-Orbitrap ELITE (Thermo-Fisher Scientific) using a Nanospray Flex source with the ion transfer capillary temperature of the mass spectrometer set at 225° C., and the spray voltage was set at 1.75 kV. Data were acquired with an approach known as an Nth Order Double Play created in Xcalibur v2.2. Scan event one of the method obtained an FTMS MS1 scan (normal mass range; 240,000 resolution, full scan type, positive polarity, profile data type) for the range 300-2000 m/z. Scan event two obtained ITMS MS2 scans (normal mass range, rapid scan rate, centroid data type) on up to twenty peaks that had a minimum signal threshold of 5,000 counts from scan event one. The lock mass option was enabled (0% lock mass abundance) using the 371.1012 m/z polysiloxane peak as an internal calibrant standard.

LCMS Data Analysis. Proteome Discoverer v1.4.1.14 (ThermoFisher) was used to analyze the data. EMBL-CDS entries corresponding to the Feb. 27, 2018 version of UniprotKB Lactobacillus rhamnosus (strain ATCC 53103/GG) UniParc sequences (proteome ID UP000000955) were used in Mascot v2.5.1 (Matrix Science Inc, Boston, Massachusetts, United States of America) and SequestHT searches. The enzyme specified was trypsin (maximum two missed cleavages with inhibition by P) with Carbamidomethyl (C) as a static modification and Oxidation(M) as dynamic. Fragment tolerance was 1.0 Da (monoisotopic) and parent tolerance was 50 ppm (monoisotopic). A Target Decoy PSM Validator node was included in the Proteome Discoverer workflow. The result files from Proteome Discoverer were loaded into Scaffold Q+S v4.4.5 (Proteome Software Inc, Portland, Oregon, United States of America). Scaffold was used to calculate the false discovery rate using the Scaffold Local FDR and Protein Prophet algorithms. Peptides were accepted if the identification had probability greater than 99.9% and parent mass error within 2 ppm. Proteins were accepted if they had a probability greater than 99.9% and at least one peptide. Proteins were grouped into clusters to satisfy the parsimony principle.

### Example 1

### Lactobacillus rhamnosus GGProduces Exosome-Like Nanoparticles

The sizes and concentrations of nanoparticles produced by LGG were characterized. LGG-derived NPs (LDNPs) were isolated by ultracentrifugation from bacteria culture (2×10⁹CFU/ml). The mean diameter of LDNPs was 75±12.7 nm, and the protein concentration of LDNPs preparation was 2.43±0.45 μg/ml. Numerous proteins were identified in LDNPs, some of which are presented in Table 2.

TABLE 2
Exemplary Proteins Identified in Isolated LDNPs

| | | Quan- |
|---|---|---|
| | | titative |
| Gene Name | Symbol | Value* |
| Cell wall-associated glycoside hydrolase | p75 | 1.55 × 10⁸ |
| Glyceraldehyde-3-phosphate dehydrogenase | gapA | 6.23 × 10⁸ |
| Surface antigen (NLP/P60) | LGG_02016 | 2.57 × 10⁸ |
| ABC transporter, sugar transporter | malE | 1.96 × 10⁷ |
| periplasmic component | | |
| Cell envelope-associated proteinase, | prtR2 | 1.27 × 10⁷ |
| lactocepin PrtR | | |
| Phage-related minor capsid protein (GpG | gpG | 1.25 × 10⁷ |
| protein) | | |
| Surface antigen | p40 | 1.3 × 10⁷ |
| Aminopeptidase C | pepC2 | 1.0 × 10⁷ |
| Conserved protein | LGG_00574 | 8086600 |
| Adhesion exoprotein | LGG_02923 | 6632800 |
| Elongation factor Tu (EF-TU) | tuf | 6294200 |
| Pilus specific protein, ancillary protein | spaC | 5988900 |
| involved in mucus-adhesion, contains von | | |
| Willebrand factor (VWF) domain | | |
| ABC transporter, oligopeptide-binding protein | oppA | 5315900 |
| ABC transporter, oligopeptide-binding protein | oppA | 5257600 |
| ABC transporter, substrate-binding protein | ABC-SBP | 5091700 |
| Conserved protein | LGG_00790 | 4913500 |
| Enolase | eno | 3807100 |
| LSU/50S ribosomal protein L7/L12P | rplL | 3264100 |
| 60 kDa chaperonin GROEL | groL | 3201400 |
| DNA-binding protein HU | hup | 2855900 |
| Cell envelope-related transcriptional attenuator | wzr1 | 2817700 |
| Penicillin-binding protein 1A | pbp1A | 2493500 |
| Conserved extracellular matrix binding protein | LGG_01865 | 2314000 |
| Putative protein without homology | LGG_02225 | 2277300 |
| Tagatose 1,6-diphosphate aldolase | lacD | 2136000 |
| PTS system, mannose-specific IID component | manD | 2047700 |
| Phage-related prohead protease | LGG_02901 | 1977400 |
| 10 kDa chaperonin GROES | groS | 1908600 |
| Cell surface protein | yqcC | 1833300 |
| Pilus specific protein, major backbone protein | spa | 1710300 |
| NADH peroxidase | npr | 1699800 |
| Conserved protein | LGG_00721 | 1524900 |
| L-lactate dehydrogenase | ldh | 1497700 |
| Conserved protein | lhv | 1338300 |
| Conserved protein | LGG_00673 | 1044400 |
| Putative protein without homology | LGG_01093 | 1039300 |
| Conserved protein | LGG_01782 | 894960 |
| Pyruvate kinase | pyk | 890840 |
| LSU/50S ribosomal protein L11P | rplK | 801230 |
| Cold shock protein | cspC | 556960 |
| ABC transporter, phosphate-binding protein | pstS | 523290 |
| Triosephosphate isomerase | tpiA | 518850 |
| Conserved protein | LGG_00583 | 434590 |
| PTS system, mannose-specific IIAB component | manA | 429830 |
| D-alanyl-D-alanine carboxypeptidase | dacA | 346980 |
| N-acetylmuramoyl-L-alanine amidase | ami | 337860 |
| 2-dehydro-3-deoxyphosphogluconate | eda | 319470 |
| aldolase/4-hydroxy-2-oxoglutarate aldolase | | |
| Conserved protein | LGG_00116 | 256450 |
| ABC transporter, substrate-binding protein, | metQ | 173380 |
| NLPA lipoprotein | | |
| Lipoprotein (pheromone precursor) | cad | 151220 |
| Fructose-bisphosphate aldolase | fba | 124390 |
| Cold shock protein | cspA | 115030 |
| ATP synthase A chain | atpA | 100830 |
| ATP synthase B chain | atpD | 69909 |
| Phage-related major tail protein | LGG_01134 | 43632 |
| Manganese-dependent inorganic | ppaC | 35815 |
| pyrophosphatase | | |
| Beta-N-acetylhexosaminidase (GH3) | nagZ | kDa |
| | | 7,902.60 |

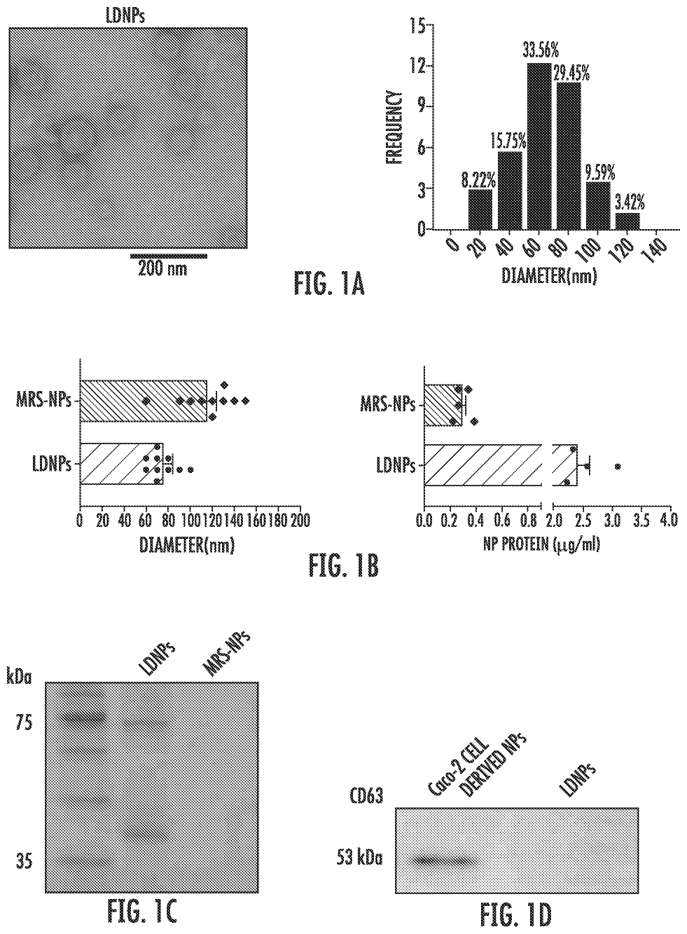

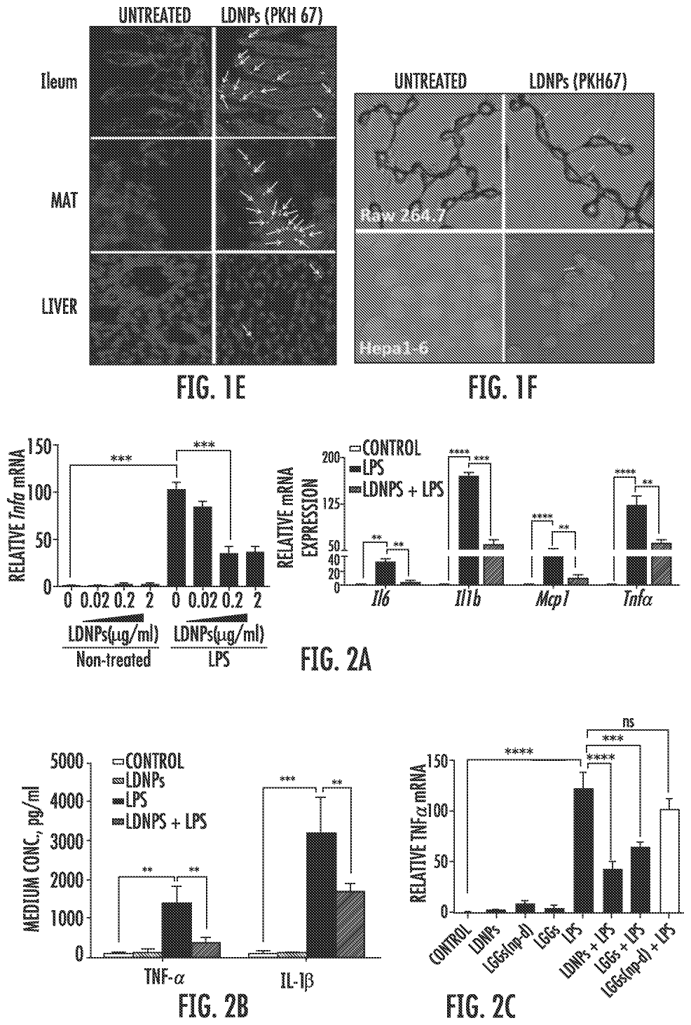

The NPs from culture medium without bacterial inoculation were also analyzed. MRS medium (for LGG culture) contained NPs with a diameter of 115±26.4 nm, which is larger than LDNPs isolated from LGG-conditioned culture supernatant. In addition, the protein concentration of NPs from the medium (0.30±0.028 μg/ml) were significantly lower compared to LDNPs (2.43±0.45 μg/ml) (FIGS. 1A and 1B). SDS-PAGE analysis showed that there were no specific protein bands in MRS-derived NPs. In contrast, LDNPs showed significant protein bands at sizes of 75 and 40 159 kDa (FIG. 1C). Proteomics analysis of LDNPs identified 60 proteins consisting of those involved in metabolism, cell wall component/peptidoglycan remodeling, transporters, structure components of ribosomes, nucleic acid binding proteins, phage related proteins and amidases. Of which, p75 and p40 abundantly existed in the LDNPs. Previous study has shown that LGG produced the signature protein p75 and p40 (Yan et al. (2007) Soluble proteins produced by probiotic bacteria regulate intestinal epithelial cell survival and growth. Gastroenterology 132:562-575). The strong Coomassie Blue staining of the proteins from LDNPs at 75 and 40 kDa are thus likely p75 and p40 proteins, respectively. Proteins in LDNPs seem stable in the solution with pH of 2.2, because incubation of the LDNPs in a pH 2.2 solution at 37° C. for 2 hours did not cause degradation or aggregation of p75 and p40 proteins (FIG. 24). Furthermore, there was no positive immune-staining of the CD63 band in LDNPs (FIG. 1D), indicating that the LDNPs do not contain eukaryotic ELNPs.

Which mouse organs and/or cells took up LDNPs when administered orally was also investigated. LDNPs were labeled with fluorescent dye PKH67 and gavaged to mice. Sliced tissues showed that LDNPs localized abundantly in the intestine villi, lamina propria of ileum and mesenteric adipose tissue, but rarely in the liver (FIG. 1E). Macrophage RAW264.7 cells and mouse hepatocyte Hepa1-6 cells were incubated with PKH67-labeled LDNPs. PKH67-positive LDNPs were found in macrophages but not hepatocytes (FIG. 1F). These data indicated that LDNPs are taken up mainly by the intestine immune and epithelial cells when given orally.

### Example 2

### LDNPs Inhibit LPS-induced Inflammation in Macrophage

To investigate the inflammatory response, RAW264.7 cells, peritoneal macrophages (PMs), and bone marrow-derived macrophages (BMDMs) were pretreated with LDNPs and then stimulated by LPS. LPS significantly upregulated the mRNA expression of Tnfa. LDNP at 0.02 μg/ml slightly, and at 0.2 μg/ml significantly suppressed Tnfα mRNA expression in RAW264.7 cells. A higher concentration of LDNPs (2 μg/ml) had no further effect (FIG. 2A, left panel). 0.2 μg/ml LDNPs were employed in experiments hereinafter. In addition to Tnfa, LDNP pretreatment also suppressed LPS-induced mRNA expression of pro-inflammatory mediators, Il6, Il1b, and Mcp1 (FIG. 2A, right panel). LPS-induced increases in TNFα and IL1β proteins were also significantly suppressed by LDNP treatment (FIG. 2B). As disclosed herein, LGG culture supernatant suppressed LPS-induced TNFα expression (see also Wang et al. (2013) Lactobacillus rhamnosus GG reduces hepatic TNFalpha production and inflammation in chronic alcohol-induced liver injury. J Nutr Biochem 24:1609-1615).

To determine whether this effect was mediated by LDNPs, we treated RAW264.7 cells with LGG supernatant (LGGs), LDNPs or LDNP-depleted supernatant (LGGs(np-d)). LPS-induced Tnfα mRNA expression was markedly reduced by LDNPs and LGGs, but not by LGGs(np-d) (FIG. 2C), suggesting that LDNPs mediated the inhibitory effect of LGGs on LPS-induced TNFα expression. Furthermore, as shown in FIG. 2D, the LDNPs-mediated reduction of the LPS-induced mRNA expression of Tnfα and Il1b was time-dependent. Maximal inhibition was achieved at 24-hour after the addition of LDNPs for Tnfα and at 48-hour for Il1b.

Thus, a 24-hour incubation time was employed for the subsequent experiments. The experiments in RAW264.7 cells were extended to PMs and BMDMs. Consistent with the findings in RAW264.7 cells, LDNPs significantly reduced Tnfα and Il1b mRNA expression in PM and BMDM (FIGS. 2E and 2F). It is possible that nanoparticles from other bacteria could also interact with LPS directly, thus having an impact on LPS-induced inflammatory response. To confirm that the inhibition of inflammation in macrophages by LDNP was LGG-specific, the same experiments were performed using Bilophila wadsworthia-derived nanoparticles. No such effects were observed.

### Example 3

### LDNPs Treatment Prevents LPS-Induced Sepsis

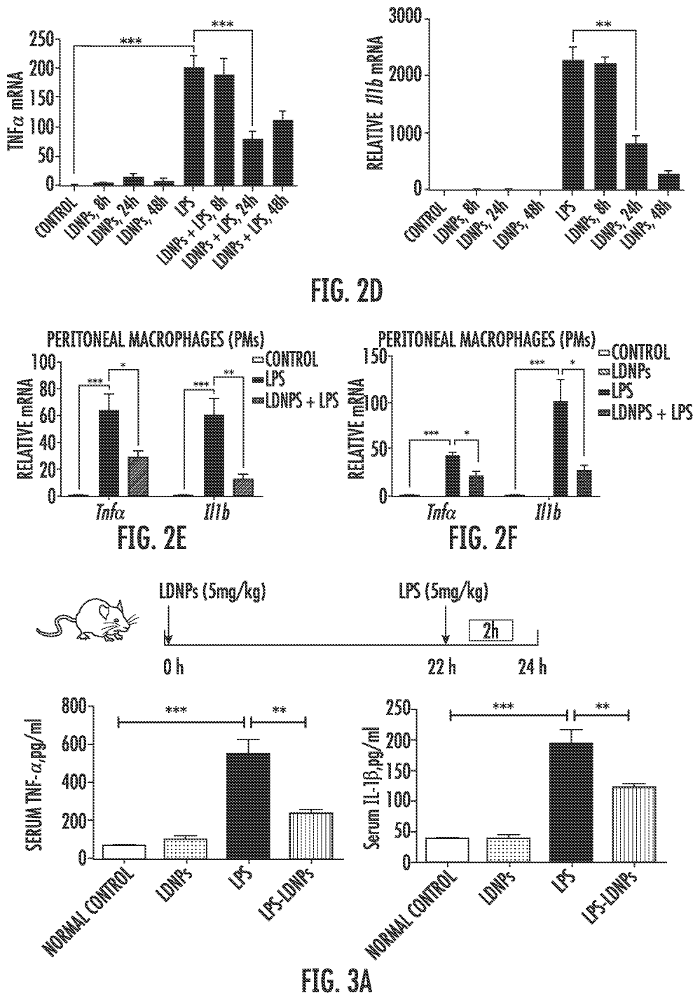

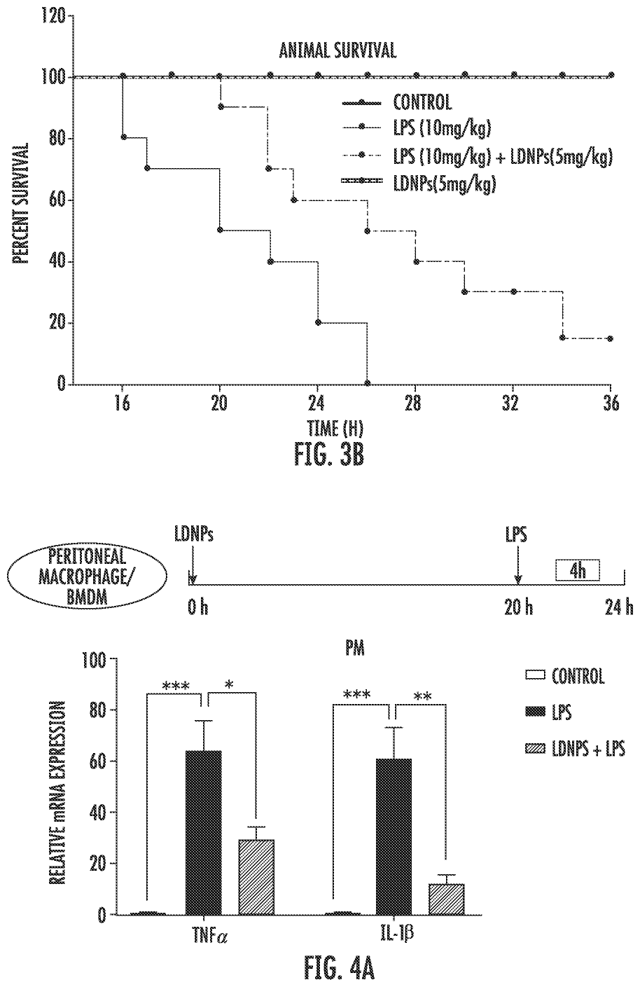

Female C57BL/6 mice were treated with LDNPs (5 mg/kg) by I.P. injection and, 22 hours later, the mice were I.P. injected LPS (5 mg/kg) as depicted in the top panel of FIG. 3A. After 2 hours, mice were sacrificed and serum levels of pro-inflammatory cytokine TNF-α and IL-1β were assayed. As shown in the bottom panel of FIG. 3A, serum TNF-α and IL-1β were both significantly reduced by treatment with LDNPs.

Survival rates between control and LPS groups with or without LDNPs pretreatment were also determined. Female C57BL/6 mice were divided into 4 groups, mice were pretreated with LDNPs at 5 mg/kg for 24 hours and LPS 10 mg/kg by I.P. injection. As shown in FIG. 3B, mice treated with LPS that had been pretreated with LDNPs showed increased survival as compared to mice that did not receive the pretreatment.

### Example 4

### LDNPs Inhibits LPS-Induced Pro-Inflammatory Cytokine Expression in Peritoneal Macrophages and Bone Marrow-Derived Macrophages

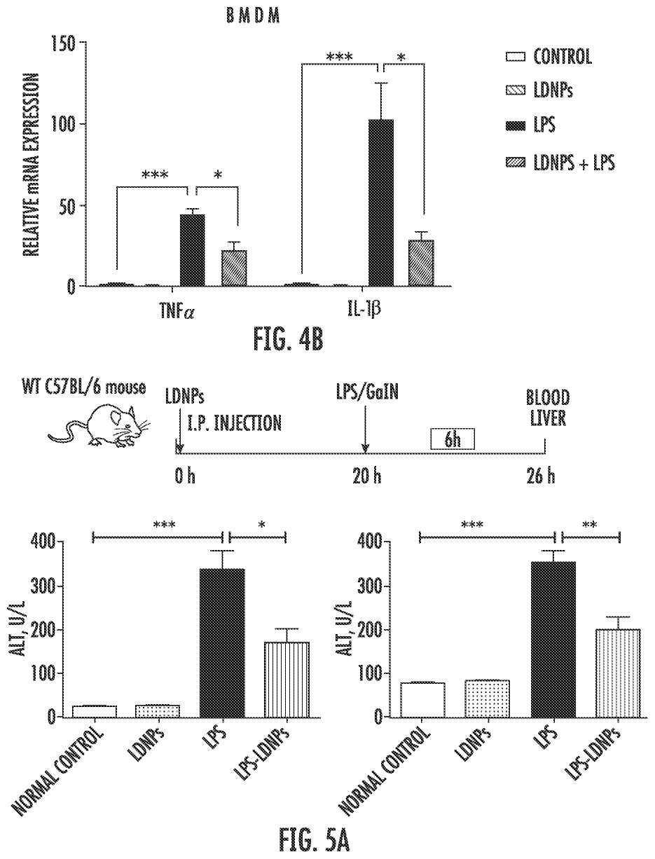

Macrophages were incubated with LDNPs (0.2 μg protein/ml) for 20 hours, after which LPS (100 ng/ml) was added to the culture for 4 hours (depicted in the top panel of the FIG. 4A). TNFα and IL-β mRNA levels were analyzed in peritoneal macrophages (PM) and bone marrow-derived macrophages (BMDM). Treatment with LDNPs significantly reduced TNFα and IL-P mRNA levels in both PM (FIG. 4A) and BMDM (FIG. 4B).

### Example 5

### LDNPs Prevents LPS/GalN-Induced Acute Liver Failure

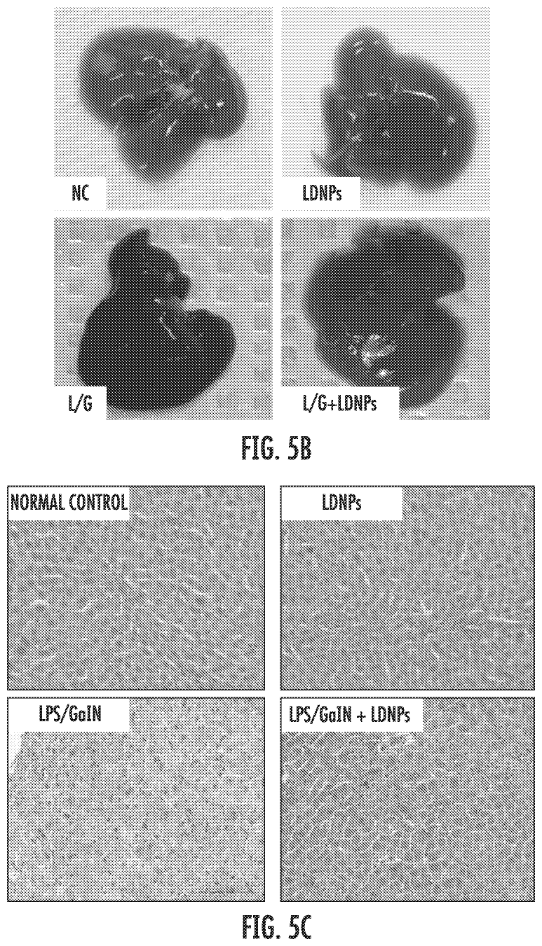

Female C57BL/6 mice were treated with LDNPs (5 mg/kg) via I.P. injection for 26 hours, the last 6 hours of which included LPS (50 μg/kg) and GalN (300 mg/kg) treatment by intraperitoneal injection as depicted in the top panel of the FIG. 5A. The levels of ALT and AST in serum were assayed. As shown in FIG. 5A, serum levels of both ALT and AST were significantly reduced by treatment with LDNPs.

The gross morphologies of livers of treated mice were also examined, representative photographs of which are depicted in FIG. 5B, FIG. 5B shows that LDNP treatment preserved liver overall morphology.

Liver sections were also stained with hematoxylin and eosin (H&E). As shown in FIG. 5C, liver architecture was well preserved in LDNPs pretreatment group.

### Example 6

### LDNPs Protect Against LPS/GalN-Induced Hepatic Apoptosis

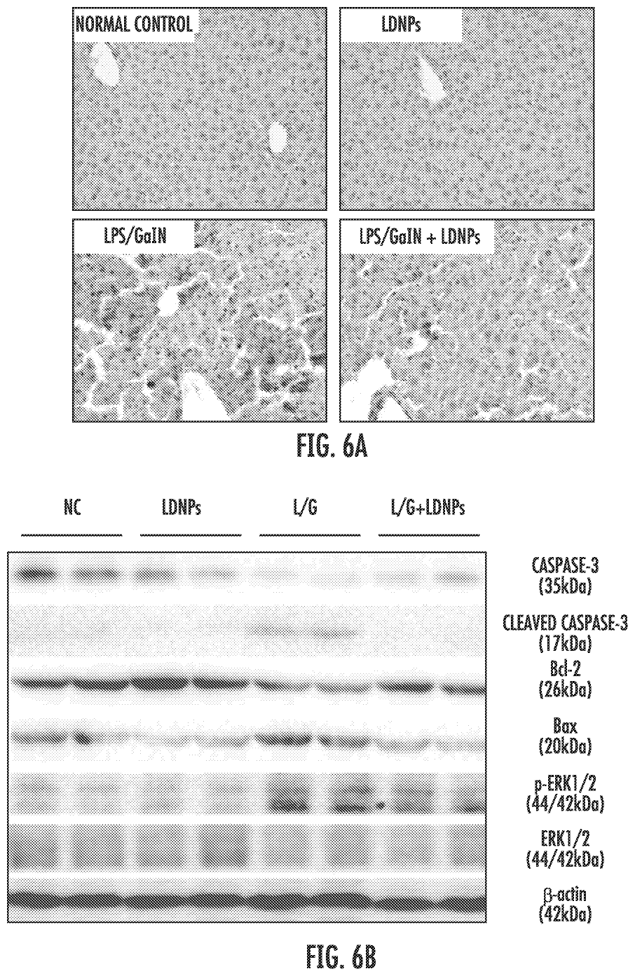

Hepatic apoptosis was also examined by TUNEL staining in LPS/GalN-treated animals with or without treatment with LDNPs. A series of representative photomicrographs of TUNEL staining of liver sections of these animals is shown in FIG. 6A. As seen therein, LPS/GalN induced a large degree of apoptosis (dark staining in the lower left panel of FIG. 6A), whereas LDNPs did not induce apoptosis (lack of dark staining in the upper right panel of FIG. 6A). Treatment with LDNPs noticeably reduced the degree of hepatic apoptosis in LPS/GalN-treated livers (relatively less dark staining in the lower right panel of FIG. 6A as compared to the lower left panel).

The presence and levels of several markers of apoptosis were assayed by immunoblot. As shown in FIG. 6B, western blotting confirmed the TUNEL staining results.

### Example 7

### LDNPs Protect Against LPS/GalN-Induced Inflammation

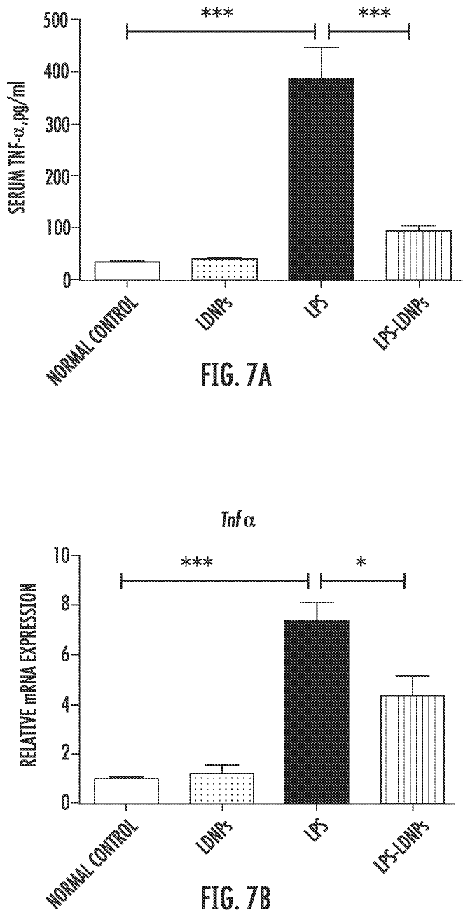

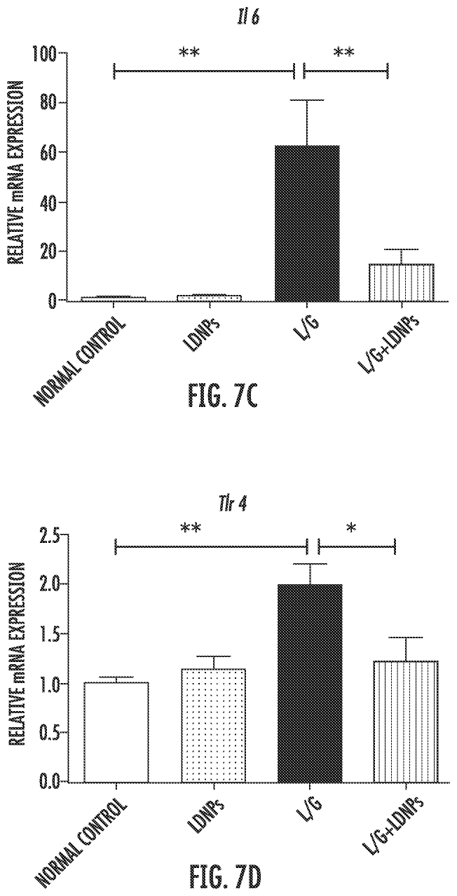

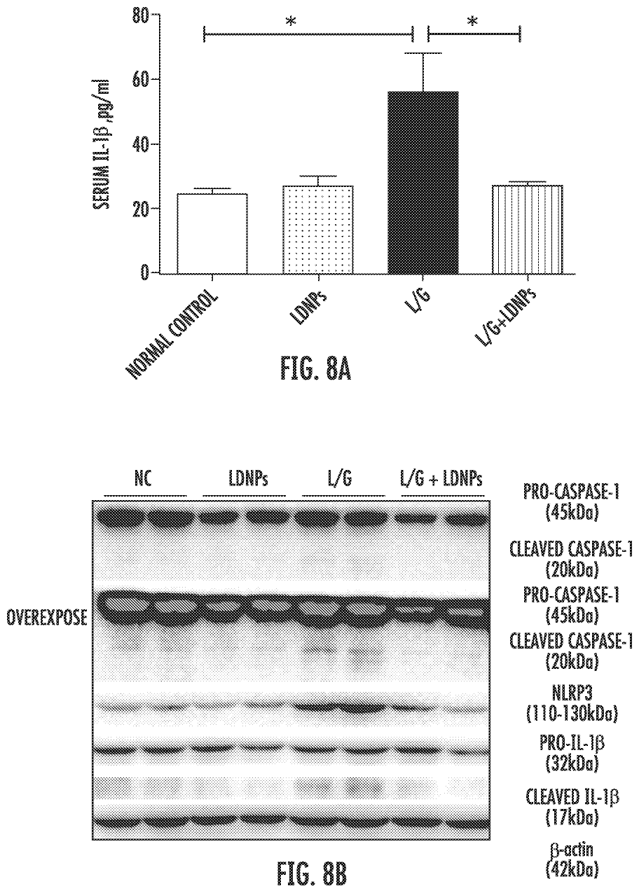

LPS-GalN-treated animals were also tested for various markers of inflammation. These markers included TNF-α, interleukin-6 (IL-6 or Il 6), and toll-like receptor 4 (Tlr 4). The results are presented in FIGS. 7A-7D.

FIG. 7A is a bar graph showing serum TNF-α protein levels in treated mice as determined by ELISA. Serum of LPS-GalN-treated animals that received LDNPs showed a significant reduction in serum TNF-α as compared to animals that did not receive LDNPs.

mRNA expression levels of these cytokines in liver were also determined. As shown in FIGS. 7B-7D, treatment with LDNPs significantly reduced relative mRNA levels of the inflammatory cytokines TNF-α (FIG. 7B) and IL6 (FIG. 7C), and of Tlr 4 (FIG. 7D) in liver tissues.

### Example 8

### LDNPs Protect Against LPS/GalN-Induced Inflammasome Activation

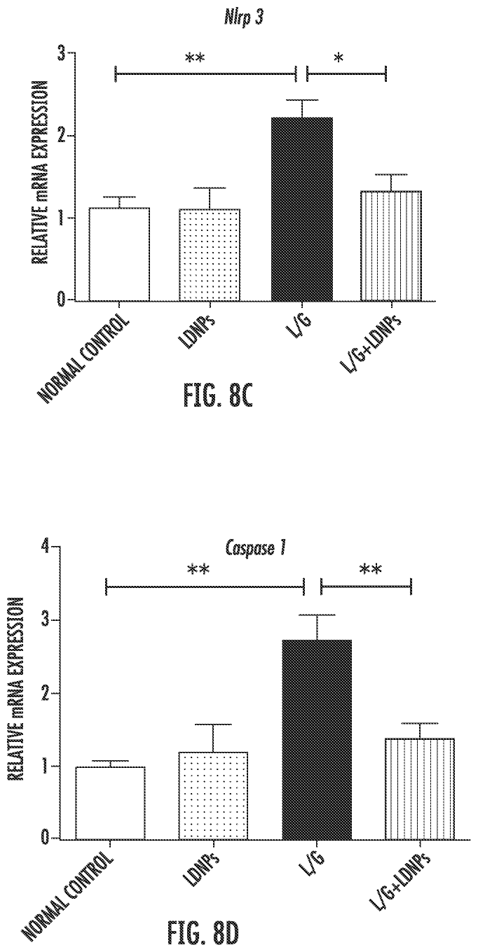

IL-1β protein levels in serum of LPS-GalN-treated animals as an indicator of inflammasome activation were determined by ELISA. As shown in in FIG. 8A, serum IL-1β protein levels were significantly reduced by treatment with LDNPs.

FIG. 8B is an immunoblot of relative protein levels of IL-1β and other markers, and FIGS. 8C-8E show the results of mRNA expression analyses of Nlrp3 (FIG. 8C), Caspase 1 (FIG. 8D), and IL-1β (FIG. 8E). LDNPs treatment significantly reduced the expression of these inflammasome activation markers as well.

### Example 9

### LDNPs Enhance Hepatic EGFR Phosphorylation

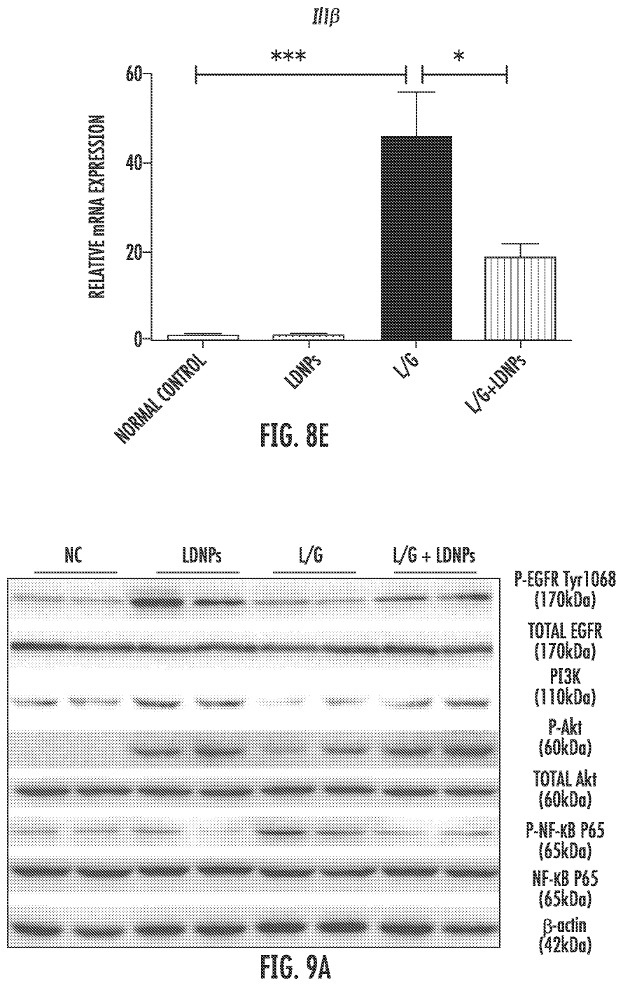

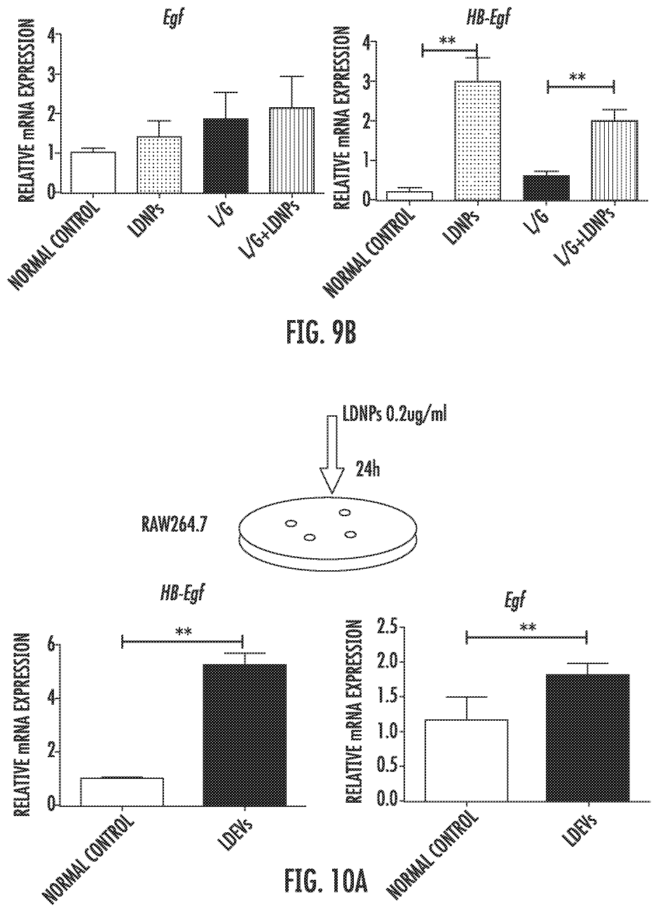

EGFR phosphorylation was assayed in LPS/GalN-treated animals with or without LDNP treatment. FIG. 9A is a representative western blot of p-EGFR, Total-EGFR, PI3K, p-Akt, total-Akt, and β-actin protein levels in liver. FIG. 9B are bar graphs of relative mRNA expression levels of EGFR ligands Egf and Hb-Egf. As shown in FIGS. 9A and 9B, LDNP treatment significantly increased the level Hb-Egf even after LPS/GalN-treatment.

### Example 10

### LDNPs Increase HB-EGF Paracrine Function in Macrophages and Hepatocytes

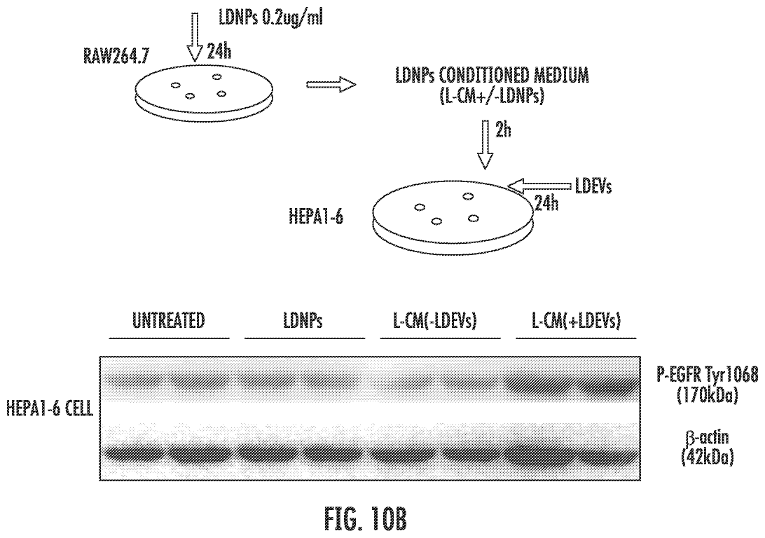

Raw264.7 cells were treated with LDNPs (0.2 μg/ml; LDEVs) for 24 hours as depicted in the top panel of the FIG. 10A. The bottom panel of FIG. 10A is a pair of bar graphs of relative mRNA levels of EGFR ligands HB-Egf and Egf as measured by RT-qPCR. A shown in FIG. 10A, treatment with LDNPs significantly increased HB-Egf mRNA expression.

Relative P-EGFR Tyr1068 protein levels in Hepa1-6 cells treated with LDNP-conditioned medium (RAW264.7 cells) for 2 hours or LDNPs for 24 hours as depicted in the top panel of the FIG. 10B were also tested by immunoblot As shown in FIG. 10B, P-EGFR Tyr1068 protein levels were increased by treatment with LDNPs (LDEVs).

### Example 11

### LDNPs Increase Paracrine Function in Peritoneal and Bone Marrow-derived Macrophages and Hepatocytes

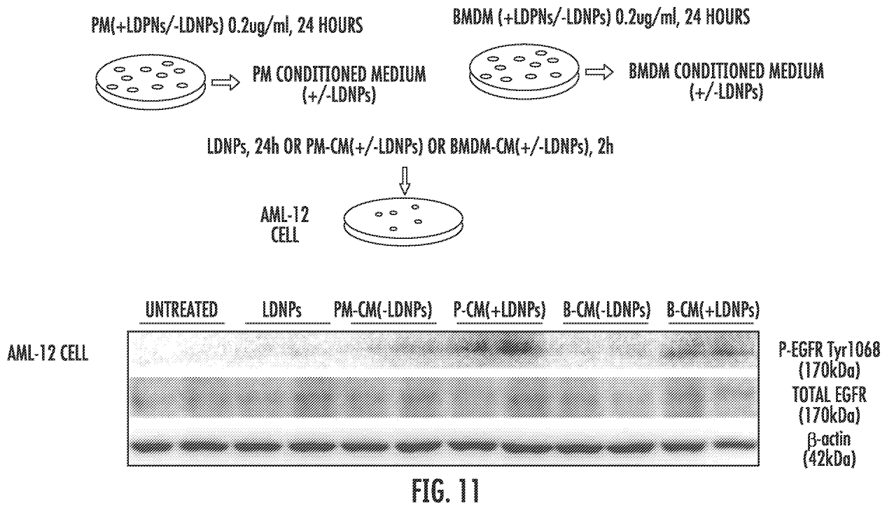

Peritoneal macrophages (PM) and bone marrow derived macrophages (BMDM) were treated with LDNPs 0.2 μg/ml for 24 hours and the conditioned medium (CM) was used to treat AML-12 cells for 2 hours as depicted in the top panel of the FIG. 11. Relative P-EGFR Tyr1068 and Total EGFR protein levels were determined by immunoblot. The bottom panel of FIG. 11 shows that conditioned medium (CM) from peritoneal macrophages (P-CM) and bone marrow-derived macrophages (B-CM) treated with LDNPs increased relative P-EGFR Tyr1068 and total EGFR protein levels.

### Example 12

### LDNPs Increased Macrophage Metalloprotease Activity

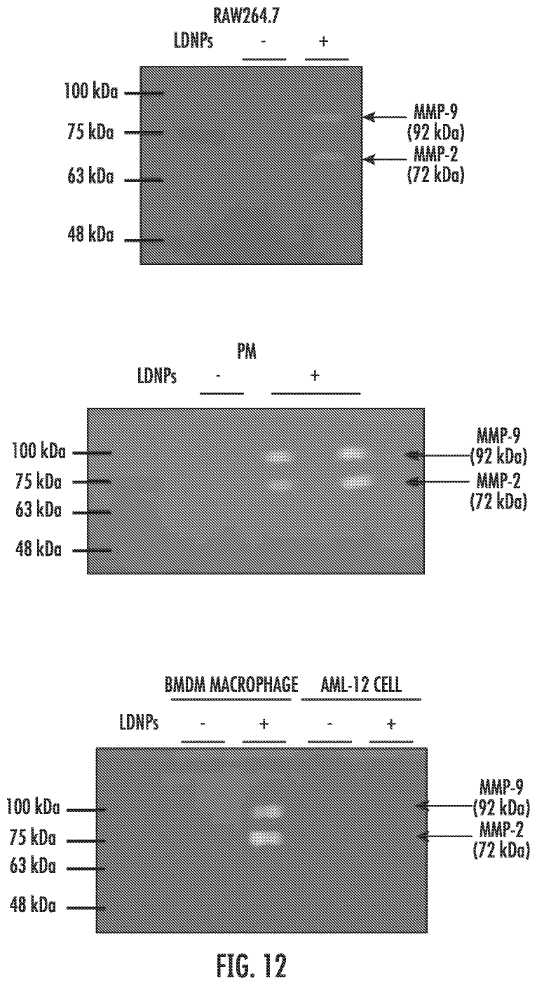

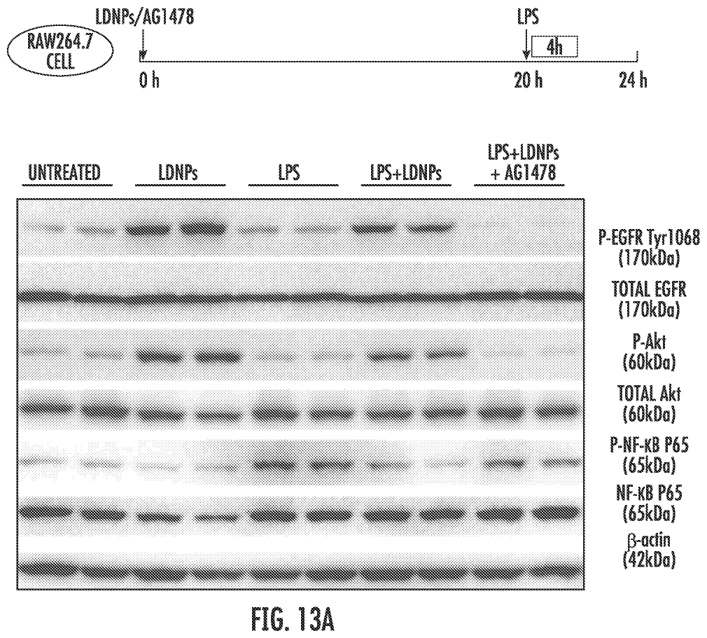

Raw264.7 cells, PMs, BMDM, and AML-12 cells were treated with LDNPs at 0.2 μg/ml for 24 hours. Activation of MMP-9 (92 kDa) and MMP-2 (72 kDa) were assayed for proteinases by zymographic analysis. As shown in FIG. 12, LDNP treatment increased the expression of macrophage metalloproteinase family members MMP-9 and MMP-2 in Raw264.7 cells, PMs, and BMDM but not AML-12 cells.

### Example 13

### LDNPs-mediated Activation of EGFR in Macrophages

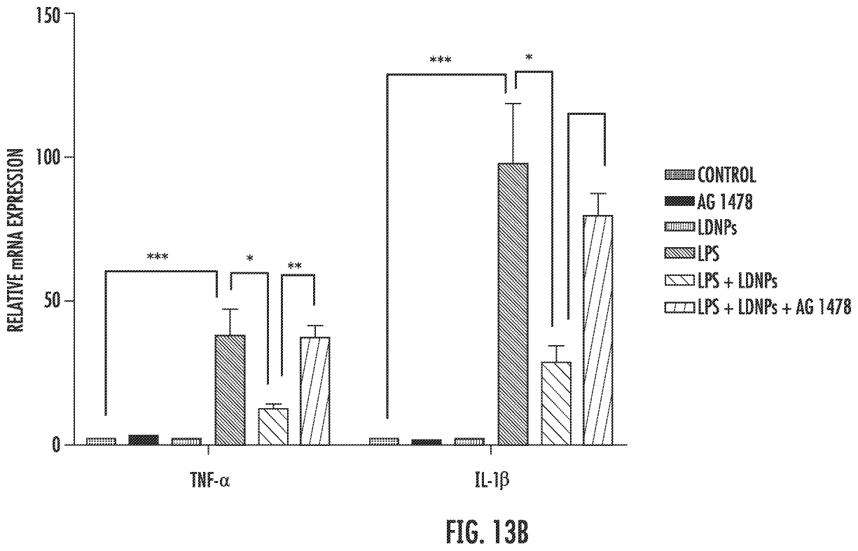

Raw264.7 cells were pretreated with LDNPs (0.2 μg/ml) or the EGFR inhibitor AG1478 (150 nM) for 20 hours and then treated with LPS for 4 hours to induce inflammation response as depicted in the top panel of the FIG. 13A. The bottom panel of FIG. 13A is a representative western blot of phosphorylated EGFR (P-EGFR Tyr 1068), Total-EGFR, PI3K, phosphorylated Akt (P-Akt), total-Akt, phosphorylated NF-κB (P-NF-κB), and β-actin showing that expression of several of these proteins were modulated by LDNP treatment.

The relative mRNA levels of pro-inflammatory cytokines TNF-α and IL-1β were also assayed, the result of which are shown in in FIG. 13B. As shown therein, LDMP treatment reduced the levels of TNF-α and IL-1β, and these reductions were inhibited by treatment with AG1478.

### Example 14

### LDNPs Stimulate Secretion of EGF in the Duodenal Brunner's Gland

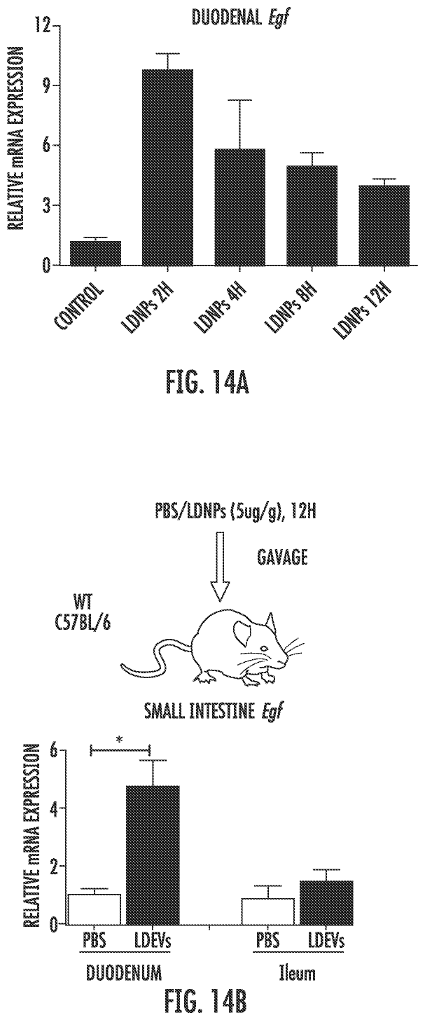

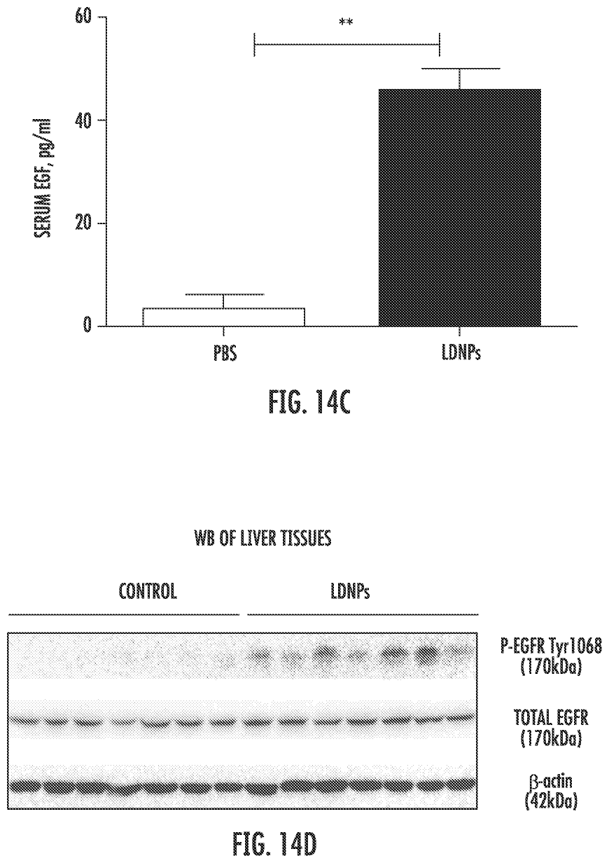

Duodenal mRNA expression levels of Egf in C57BL/6 mice treated with LDNPs (5 mg/kg) by oral gavage for various durations were tested. As shown in FIG. 14A, a time-dependent reduction in Egf expression was observed.

The relative duodenum and ileum Egf mRNA levels in C57BL/6 mice treated with LDNPs (5 mg/kg; LDEVs) by oral gavage for 12 hours as depicted in the top panel of the FIG. 14B. As set forth in the bottom panel of FIG. 14B, the relative expression of Egf was significantly increased in the duodenun but not the ileum of treated mice.

Serum levels of Egf protein were also assayed, the result of which are shown in FIG. 14C. Serum Egf protein levels were significantly increased by LDNP treatment.

Relative protein levels of phosphorylated Egfr (P-EGFR Tyr1068) and total Egfr were assayed by immunoblot. As shown in FIG. 14D, LDNPs induced the phosphorylation of Egfr in liver tissues.

### Example 15

### Effects of LDNPs Treatment on Duodenal EGF Secretion

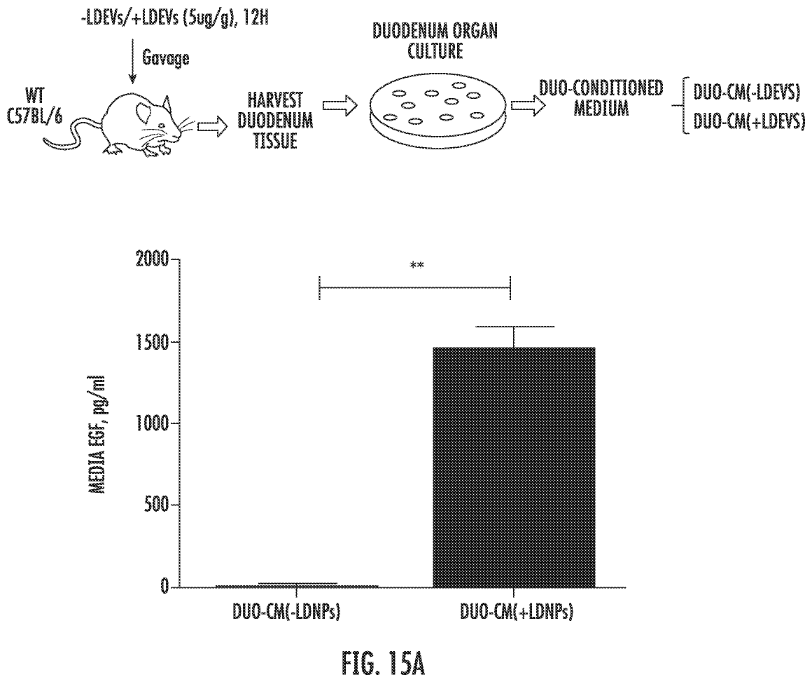

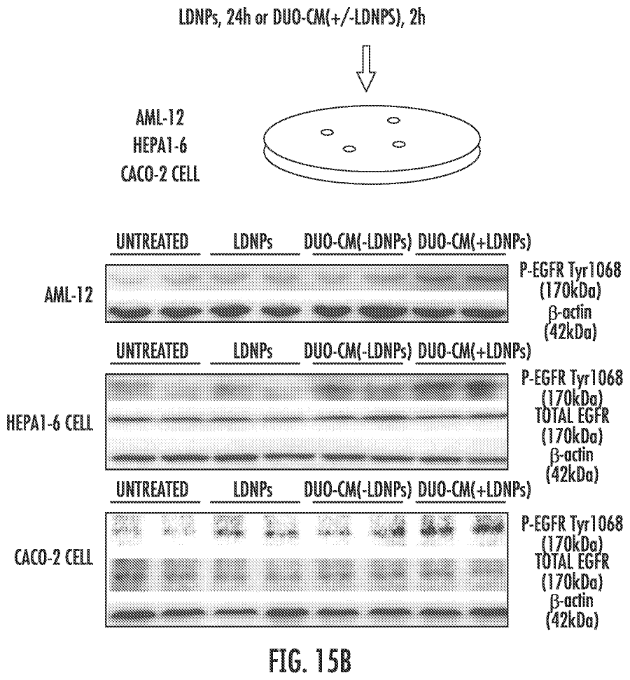

Female C57BL/6 mice were treated with LDNPs (5 mg/kg)via oral gavage for 12 hours, and the duodenum tissues were harvested for organ culture as depicted in the top panel of the FIG. 15A. As shown in FIG. 15A, EGF protein levels in duodenum organ culture medium was significantly increased by treatment with LDNPs.

LDNPs-conditioned duodenal secretions of hepatocyte EGFR signaling in AML-12, Hepa1-6, and Caco-2 cells was tested as depicted in the top panel of FIG. 15B. As shown in the bottom panel of FIG. 15B, phosphorylated Egfr (P-EGFR Tyr1068) was increased in each of these cell lines.

### Example 16

### LDNPs Improve Alcohol-Induced Steatosis

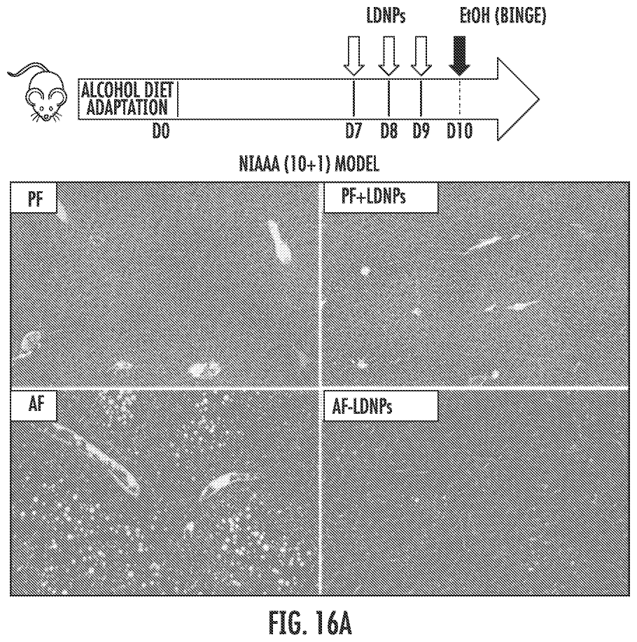

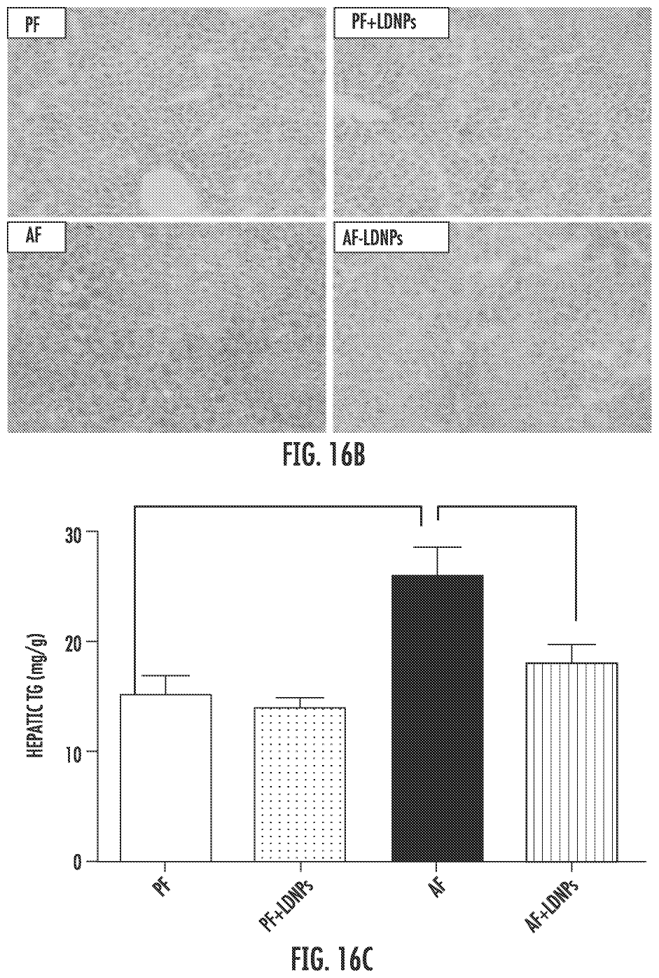

C56BL/6 mice were subjected to the normal feeding (PF: pair-fed) or an NIAAA (10d+1b) alcohol model (AF: alcohol-fed). At day 7, LDNPs were gavaged once a day for 3 days as depicted in the top panel of FIG. 16A. FIGS. 16A and 16B are representative images of H&E and oil red O staining of liver sections, respectively, showing the protective effects provided by LDNP treatment. FIG. 16C is a bar graph of hepatic levels of various triglycerides in treated mice.

### Example 17

### LDNPs Improved Alcohol-Induced Liver Injury and Hepatocyte Apoptosis

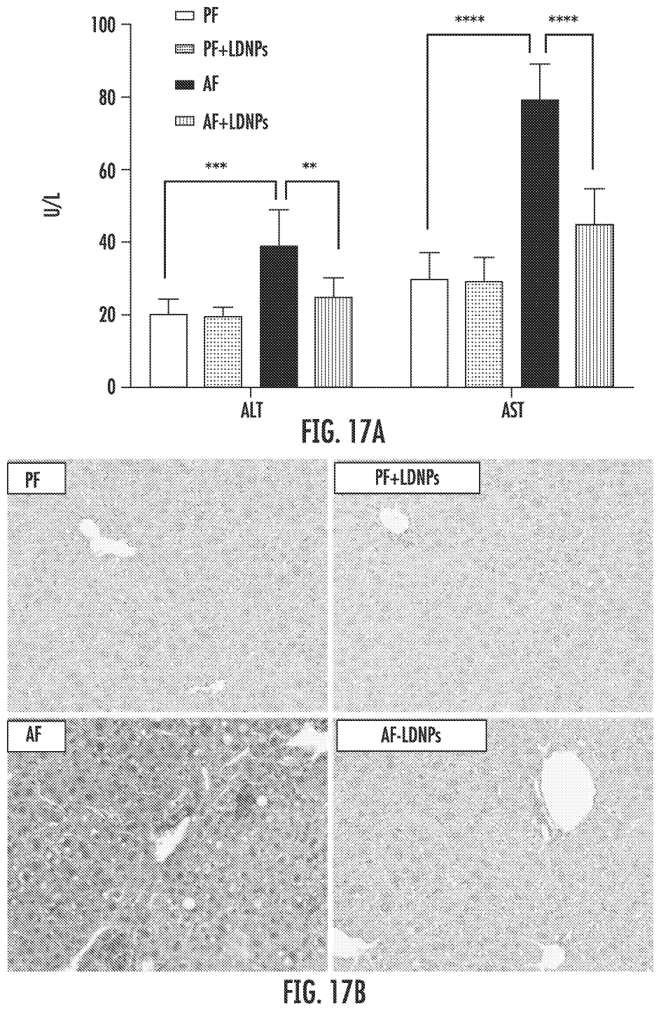

C56BL/6 mice were subjected to NIAAA (10d+1b) alcohol model. At day 7, LDNPs were gavaged once a day for 3 days as described in FIG. 16. Serum ALT and AST levels were assayed As shown in FIG. 17A, LDNP treatment reduced ALT and AST expression in both PF and AF mice. Apoptosis analysis by TUNEL staining of liver sections (FIG. 17B) showed that LDNPs protected AF liver cells from alcohol-induced apoptosis.

### Example 18

### LDNPs Increased AhR Reporter Activity and Intestinal Downstream Signaling

To investigate the potential mechanisms underlying this inhibitory effect of LDNPs on macrophages, the metabolite cargo composition of LDNPs was analyzed by LC-MS based metabolomics technologies. Over 2000 metabolites were identified. Interestingly, tryptophan-derived AhR ligands exist abundantly in the LDNP cargo, including but not limited to those shown in Table 3.

TABLE 3
Exemplary AhR Ligands Present in LDNPs

| Ligand Name | Signal Intensity |
|---|---|
| Indoieacrylic acid (IA) | 4.8 × 10⁸ |
| Indole-3-aldehyde (I3A) | 3 × 10⁷ |
| 2-Methyleneoxindole | 1.2 × 10⁷ |
| Indole | 1 × 10⁷ |
| Indole-3-lactic acid (ILA) | 8 × 10⁵ |
| Indole acetic acid | 6 × 10⁴ |

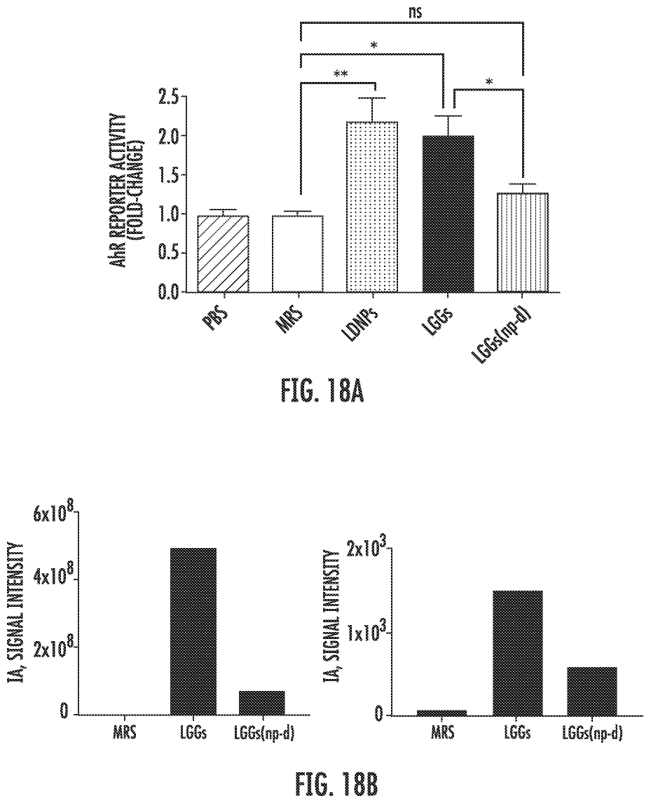

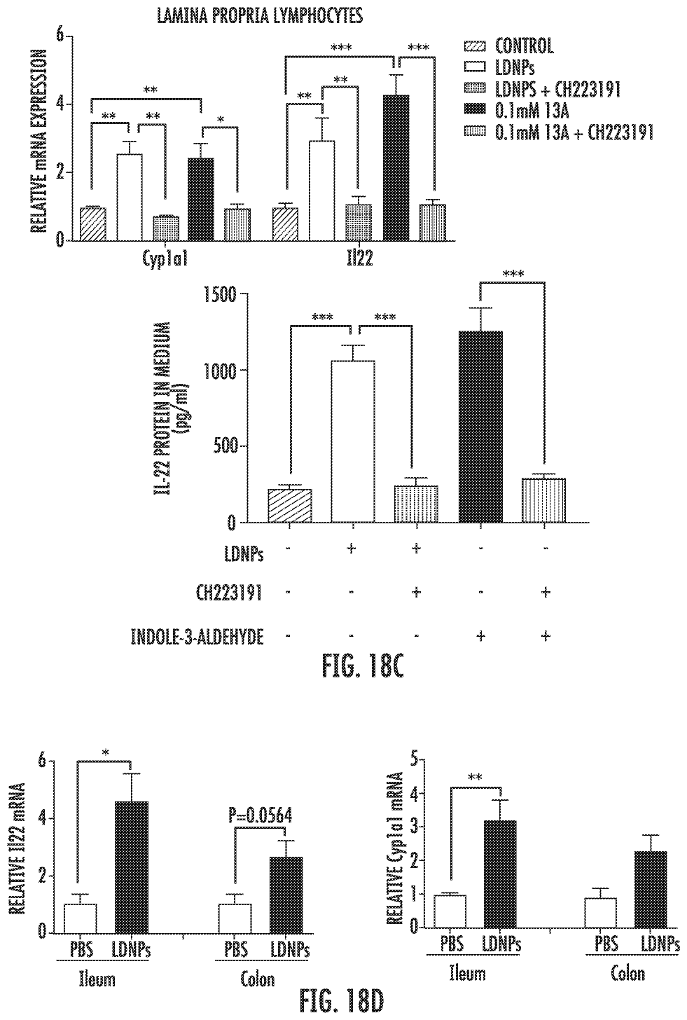

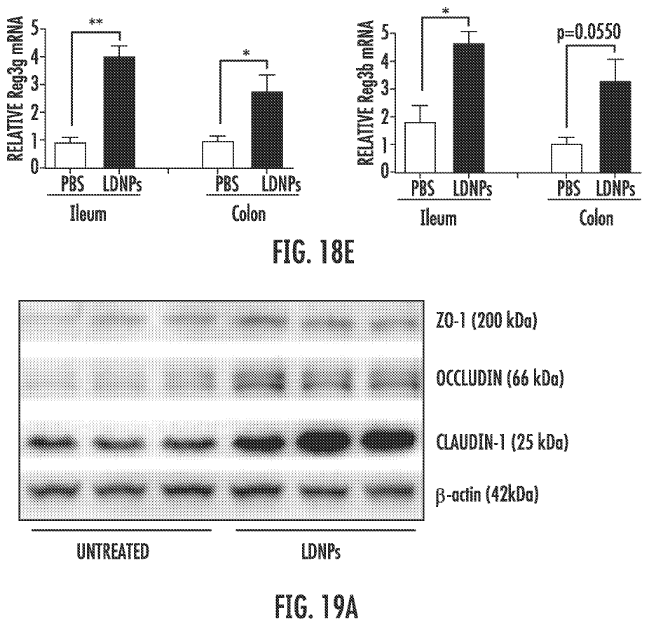

AhR reporter activity of MRS, LDNPs, LGGs, and LDNP-depleted LGGs (LGGs(np-d)) was assayed. As shown in FIG. 18A, LDNPs and LGGs induced a significant increase in reporter activity as compared to MRS or negative control (PBS), am induction that was not observed in LDNP-depleted LGGs (LGGs(np-d).

Luciferase reporter analysis showed that both LDNPs and LGGs increased AhR activity. However, when the LDNPs were depleted from LGGs, the induction of AhR reporter activity was significantly decreased (FIG. 18A). The signal intensities of IA and I3A are shown in FIG. 18B. A standard curve study by LC-MS showed a linear representation in the signal range shown on the y-axes of FIG. 18B. Moreover, when LDNPs were depleted, the concentrations of indoleacrylic acid (IA) and indole-3-aldehyde (I3A) in LGGs were markedly reduced (FIG. 18C). Taken together, these data indicated that LNDPs were enriched in AhR ligands from bacterial tryptophan metabolism.

The effects of the AhR inhibitor CH229131 on LDNPs-induced upregulation of Cyp1a1 and interleukin-22 (IL-22 or Il22) mRNA expression in lamina propria lymphocytes (LPLs) and serum protein levels in in the culture medium of LPLs were also assayed, the results of which are shown in the upper and lower panels of FIG. 18C, respectively. Significant inductions of Cyp1a1 and IL-22 were induced by LDNPs, the inductions of which were blocked by CH229131.

AhR is a ligand-activated nuclear receptor and is expressed in many cell types including intestinal type 3 innate lymphoid (ILC-3) cells. To examine whether LDNPs increase intestinal AhR signaling, Ileum lamina propria lymphocytes (LPLs) were isolated from mice. Incubation of the LPLs with LDNPs produced a significant induction of mRNA expression of Cyp1a1 and Il22, which are two transcription targets of AhR (see Cheng et al. (2015) Aryl Hydrocarbon Receptor Activity of Tryptophan Metabolites in Young Adult Mouse Colonocytes. Drug Metab Dispos 43:1536-1543.). Importantly, the effects of LDNPs on the expression of Cyp1a1 and Il22 were completely inhibited by the AhR inhibitor, CH223191 (FIG. 18C, upper panel; I3A was used as a positive control ligand for the analysis). In addition, the protein level of IL-22 in the medium was found to be increased about 5-fold by LDNPs, and the upregulation of IL-22 was completely inhibited by CH223191 (FIG. 18C, lower panel).

The relative mRNA expression of IL-22 and Cyp2E1 in mouse ileum and colon were also assayed, the results are shown in FIG. 18D. IL-22 and Cyp2E1 expression was significantly increased in the ileum by LDNPs, but the increase in colon was not quite significant. To determine whether oral administration of LDNPs increased intestinal AhR signaling, mice were gavaged with LDNPs. LDNPs administration markedly increased mRNA expression of Il22 and Cyp1a1 in the ileum, but not in the colon (FIG. 18D). It is known that intestinal IL-22 mediates the expression of Reg3b and Reg3g, two major antimicrobial peptides expressed in intestinal Paneth cells.

Relative mRNA expression levels of Reg3γ (Reg3g) and Reg3β (Reg3b) in mouse ileum and colon were also assayed. As shown in FIG. 18E, Reg3γ expression was significantly increased in the ileum and colon by LDNPs, as was Reg3β in the ileum, but the increase in colon of Reg3β was not significant. As shown in FIG. 18E, LDNP administration markedly increased ileum Reg3b and Reg3g mRNA expression. In the colon, Reg3g expression was significantly increased, whereas Reg3b was marginally increased.

### Example 19

### LDNPs Increased Intestinal Tight Junction Expression in Caco-2 Cells

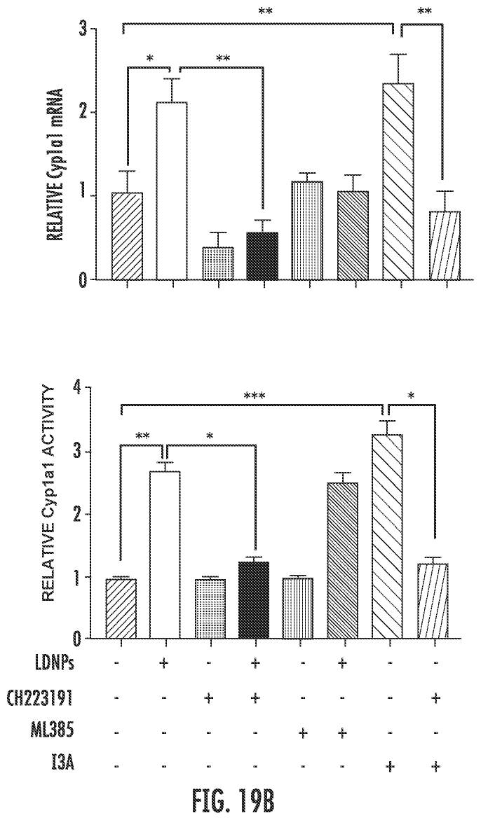

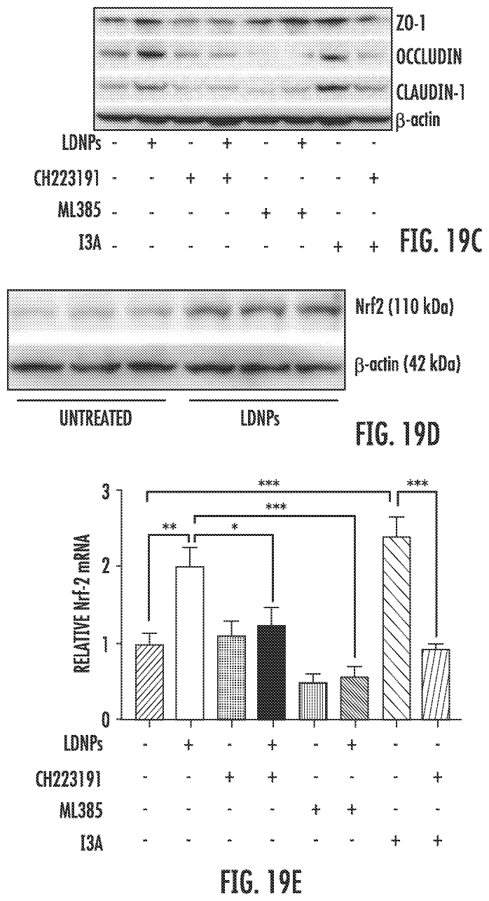

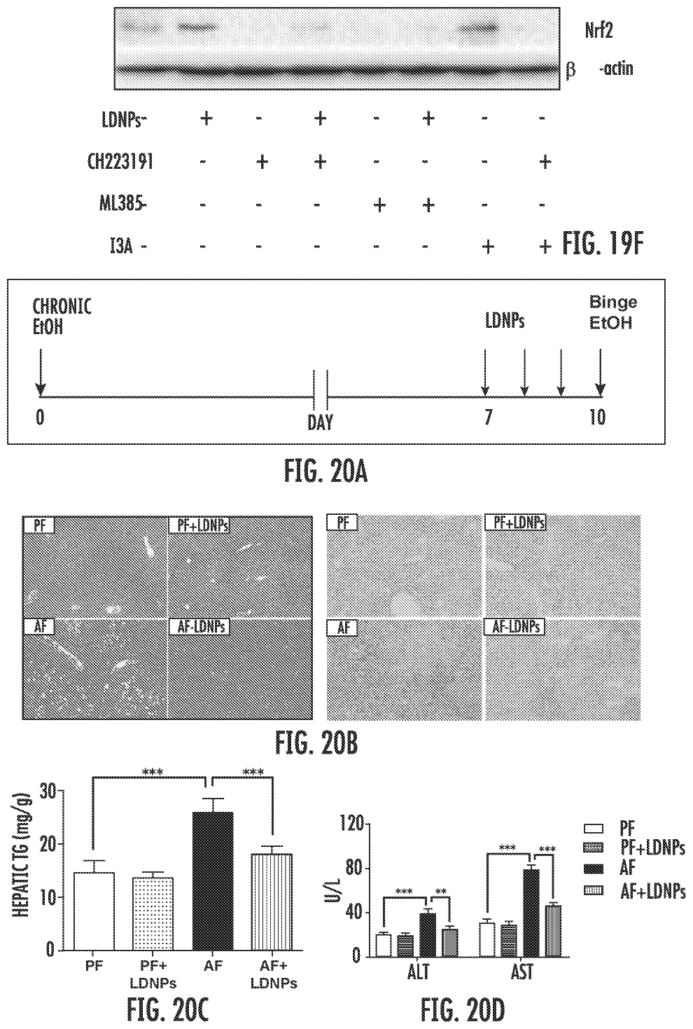

Intestinal barrier function plays a crucial role in a variety of disease conditions. To investigate whether LDNPs modulate intestinal barrier function, tight junction protein expression was assayed in intestinal epithelial cells (Caco-2). LDNP treatment significantly increased three major tight junction proteins: ZO-1, Occludin, and Claudin-1 in Caco-2 cells (FIG. 19A). FIG. 19A presents western blot analyses for ZO-1, Occludin, and Claudin-1 protein in Caco-2 cell lysates. As can be seen, each protein was induced by LDNPs.

Relative Cyp1a1 mRNA expression (upper panel of FIG. 19B) and Cyp1a1 activity (lower panel of FIG. 19B) in Caco-2 cells treated with LDNPs, CH223191 (AhR inhibitor), ML385 (Nrf2 inhibitor), and I3A were determined. LDNPs induced significant increased in both, which were blocked by CH223191.

To determine whether this effect was AhR-mediated, I3A, a ligand of AhR as a positive control, and the AhR inhibitor CH223191 were added to 241 Caco-2 cell culture. I3A markedly increased Cyp1a1 mRNA expression and Cyp1a1 activity, which was blocked by CH223191, indicating an AhR regulation. Similar to I3A, LDNPs induced a significant upregulation of Cyp1a1 mRNA expression and increased Cyp1a1 activity, which was blocked by CH223191, indicating an AhR-dependent effect of LDNPs (FIG. 19B). Importantly, both I3A and LDNPs increased the protein expression of tight junction proteins, which was inhibited by CH223191 (FIG. 19C). These data indicated that LDNPs had AhR agonist-like activity and that the upregulation of intestinal epithelial cell tight junctions by LDNPs is AhR-dependent.

Tight junction proteins have been shown to be regulated by cellular oxidative stress (Rao (2008) Oxidative stress-induced disruption of epithelial and endothelial tight junctions. Front Biosci 13:7210-7226), and Nrf2 is important in anti-oxidant regulation in the intestine (Singh et al. (2019) Enhancement of the gut barrier integrity by a microbial metabolite through the Nrf2 pathway. Nature Communications 2019:10; Wen et al. (2019) A Protective Role of the NRF2-Keap1 Pathway in Maintaining Intestinal Barrier Function. Oxid Med Cell Longev 2019:1759149).

To determine the role of oxidative stress signaling in the upregulation of tight junction proteins, a Nrf2 inhibitor, ML385, was co-administered with LDNPs. Indeed, LDNP treatment significantly increased Nrf2 expression at both mRNA and protein levels in Caco-2 cells, and this upregulation was completely inhibited by ML385 (FIG. 19D-19F). Interestingly, LDNPs-induced Nrf2 expression was also blocked by CH223191 (FIGS. 19E and 19F), suggesting that AhR was required for the upregulation of Nrf2 by LDNPs. Importantly, LDNP-induced tight junction protein upregulation was inhibited by ML385 (FIG. 19C). It should be noted that Nrf2 inhibition blocked the LDNP-induced Cyp1a1 mRNA expression but not activity (FIG. 19B).

Summarily, LDNP treatment modulated expression of several markers, and CH223191 (AhR inhibitor) and/or ML385 impacted the expression affected by LDNPs.

### Example 20

### LDNPs Prevent Alcohol-Associated Liver Disease

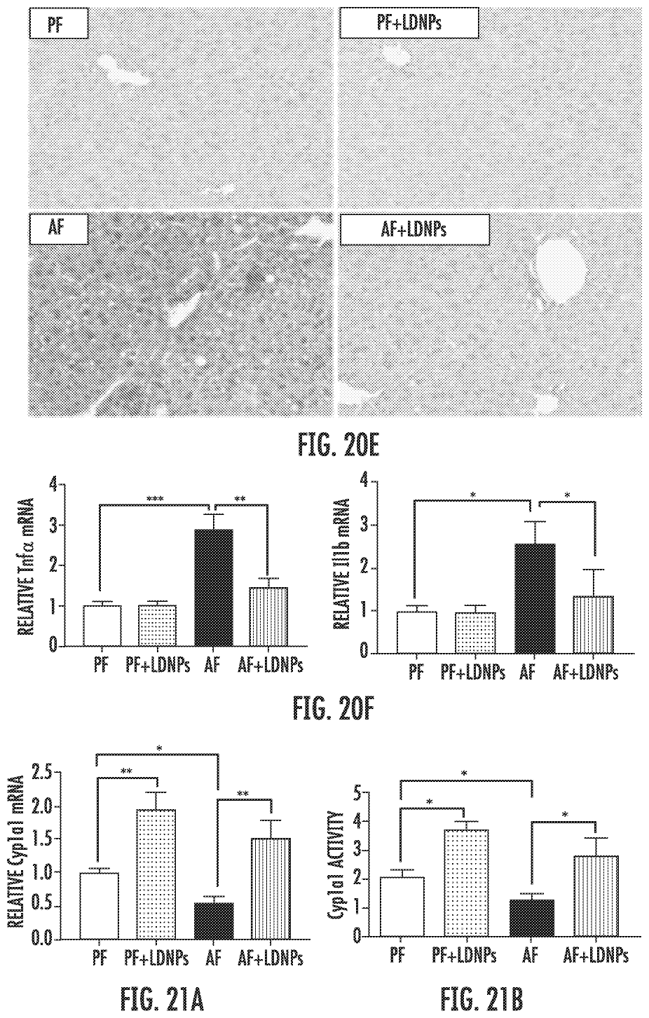

Increased serum endotoxin levels and hepatic bacterial translocation are manifestations of gut barrier dysfunction and hallmarks of ALD. To examine whether LDNP treatment could protect against alcohol-induced liver injury, mice were fed with alcohol in a binge-on-chronic alcohol exposure model. Specifically, mice were fed the Lieber DeCarli diet containing 5% EtOH (w/v) for 10 days, and a bolus of EtOH (10d+1b) was gavaged to mice on the last day, 9 hours before sacrifice. LDNPs were orally gavaged at a dose of 50 μg/mouse once a day for the last three days (FIG. 20A). Alcohol feeding increased hepatic fat accumulation, as determined by H&E and Oil Red O staining, which was markedly decreased by LDNP treatment (FIG. 20B). The histologic observations of hepatic steatosis were confirmed by hepatic triglyceride measurement (FIG. 20C). Serum levels of ALT and AST were increased by alcohol and decreased by LDNP treatment (FIG. 20D). LDNP treatment prevented the apoptotic cell death by alcohol, as demonstrated by TUNEL assay (FIG. 20E). Alcohol feeding-induced hepatic inflammation was significantly reduced by LDNPs treatment, as shown by hepatic mRNA expression of pro-inflammatory mediators, Tnfα and Il1b (FIG. 20F).

### Example 21

### LDNPs Increase Intestinal AhR Activity and Decreased Hepatic Bacterial Translocation

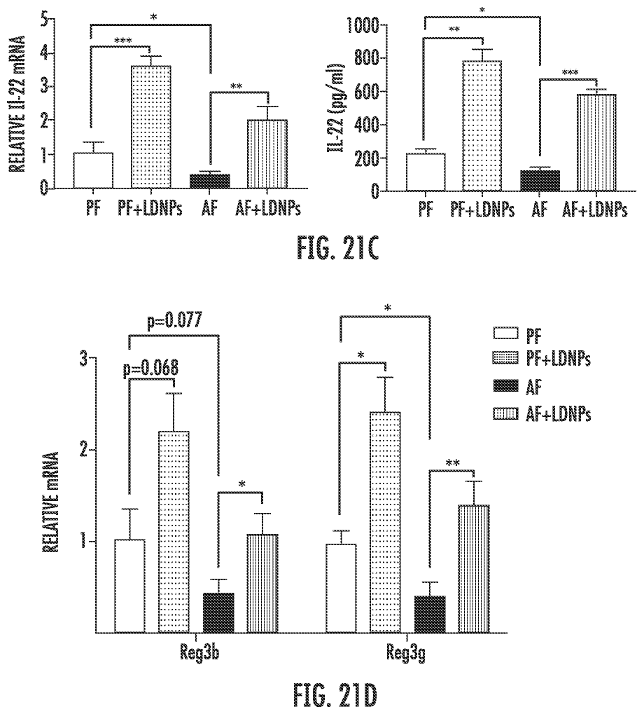

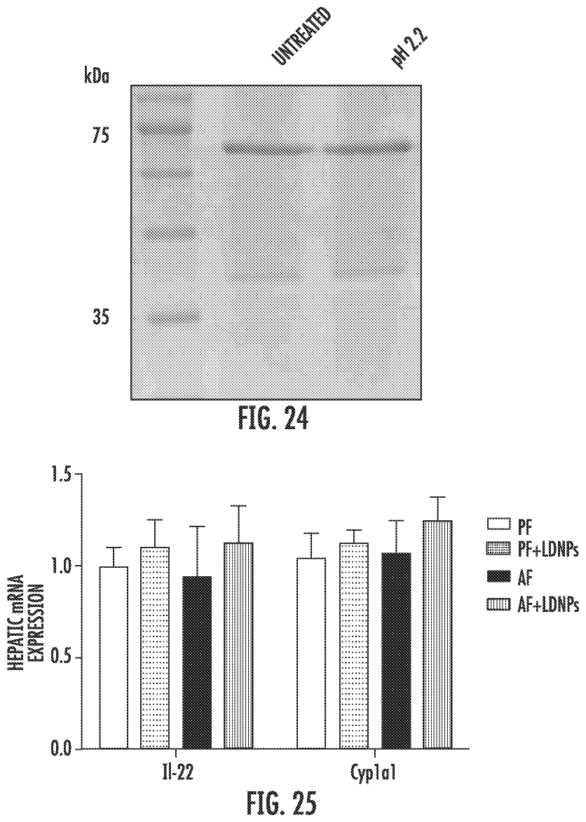

The intestinal signaling pathway linked to AhR activation in mice with experimental ALD was examined. Alcohol feeding significantly decreased intestinal Cyp1a1 mRNA expression and Cyp1a1 activity (FIGS. 21A and 21B), indicating an attenuated AhR activation by alcohol. LDNP treatment significantly increased both mRNA expression and activity of Cyp1a1 in mice under both pair feeding and alcohol feeding conditions (FIGS. 21A and 21B). Ileum mRNA and serum protein levels of IL-22 were significantly decreased by alcohol (FIG. 21C), which is consistent with another report (Hendrikx et al. (2019) Bacteria engineered to produce IL-22 in intestine induce expression of REG3G to reduce ethanol-induced liver disease in mice. Gut 68:1504-1515). Notably, LDNP treatment markedly increased ileum IL-22 expression (FIG. 21C). Importantly, no effects of either alcohol or LDNPs on IL22 and Cyp1a1 mRNA expression in hepatic tissues was observed (FIG. 25), indicating the effect of LDNPs on the AhR pathway was likely intestine-specific.

Reg3β and Reg3γ are produced in Paneth cells and play a critical role in maintaining bacterial homeostasis and inhibiting bacterial translocation. Ileum mRNA levels of Reg3b or Reg3g were slightly or significantly decreased by alcohol, respectively. LDNP treatment increased mRNA expression of Reg3b and Reg3g under both pair feeding and alcohol feeding conditions (FIG. 21D). Furthermore, lysozyme staining of ileum tissue showed decreased Paneth cell numbers, which were increased by LNDPs treatment (FIG. 21E). Elevation of Reg3β and Reg3γ should result in reduced bacterial translocation. As expected, LDNP treatment was found to markedly decrease alcohol feeding-increased hepatic bacteria load (FIG. 21F). Taken together, these results demonstrated that LDNPs increased intestinal AhR-IL-22-Reg3 signaling pathways leading to reduced bacterial translocation in the livers of mice fed alcohol.

### Example 22

### LDNP Treatment Decreases Circulating Endotoxin Levels Through Nrf2 Activation

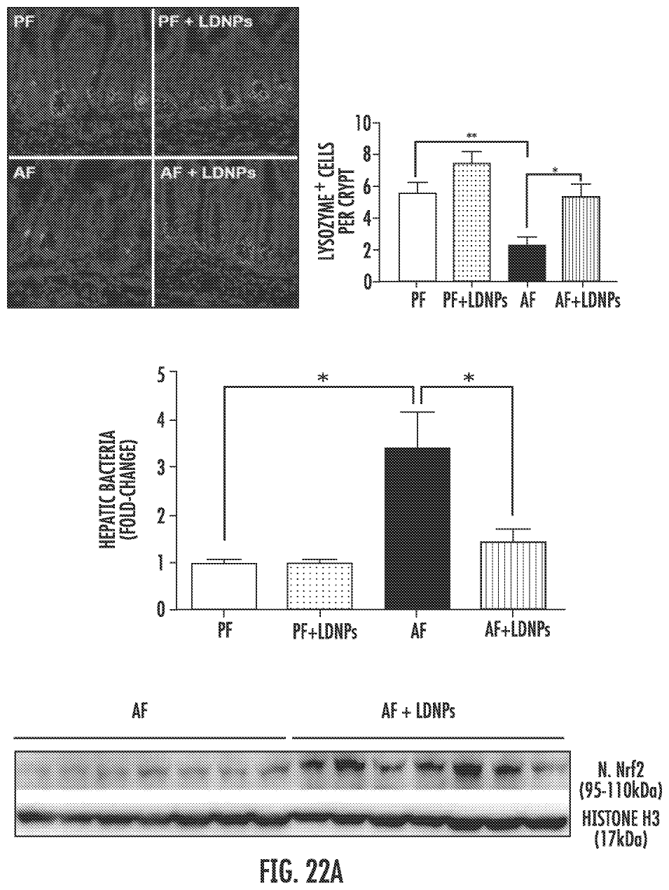

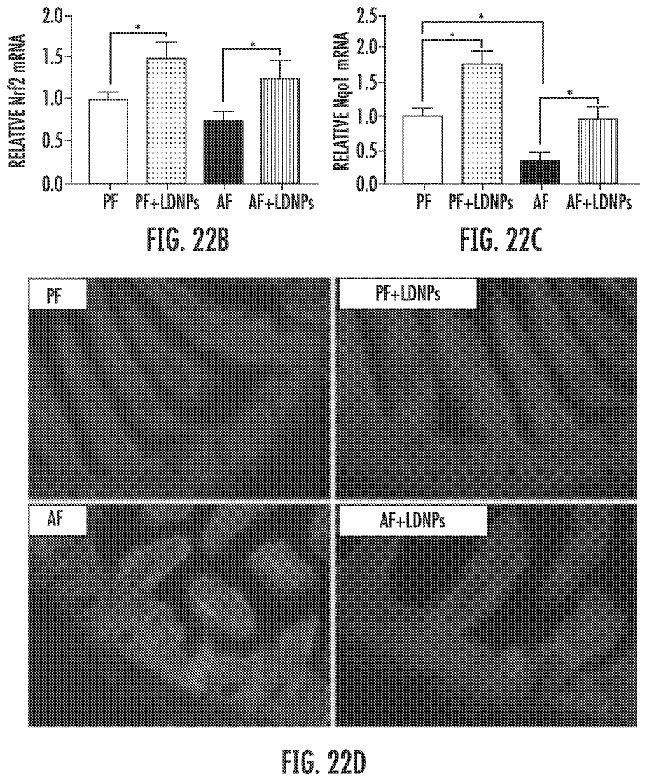

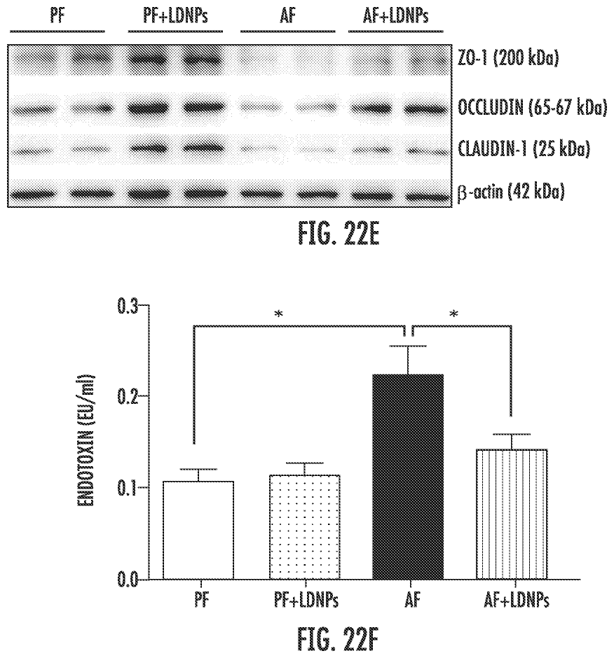

Whether LDNP treatment affected the intestinal Nrf2 pathway leading to upregulation of intestinal tight junction protein expression and reduction of endotoxemia was investigated. Alcohol feeding significantly decreased ileum nuclear Nrf2 protein levels (FIG. 22A) and slightly decreased Nrf2 mRNA expression (FIG. 22B). LDNP treatment increased intestinal Nrf2 expression in PF mice and prevented the reduction in AF mice (FIG. 22B).

NAD(P)H: quinone acceptor oxidoreductase 1 (Nqo1) is an inducible enzyme that is regulated by the Nrf2 pathway and plays an important role in combating oxidative stress (Lau et al. (2015) Role of Nrf2 dysfunction in uremia-associated intestinal inflammation and epithelial barrier disruption. Dig Dis Sci 60:1215-1222). Alcohol feeding significantly decreased intestinal Nqo1 mRNA expression. LDNP treatment markedly increased Nqo1 mRNA in PF mice and prevented the reduction in AF mice (FIG. 22C).

Cellular ROS status was also investigated. DHE staining of the ileum tissues showed that alcohol feeding increased ROS, which was significantly reduced by LDNP treatment (FIG. 22D).

Intestinal leakiness was then evaluated by examining the intestinal tight junction protein expression and serum endotoxin (LPS) levels. Agreeing with a previous study, alcohol feeding decreased ileum protein expression of ZO-1, Occludin and Claudin-1 (see Wang et al. (2013) Lactobacillus rhamnosus GG reduces hepatic TNFalpha production and inflammation in chronic alcohol-induced liver injury. J Nutr Biochem 24:1609-1615). LDNP treatment prevented the downregulation of these proteins (FIG. 22E). Alcohol feeding increased serum LPS levels, which was inhibited by LDNP treatment (FIG. 22F). Taken together, these data demonstrated that LDNPs administration inhibited alcohol exposure-induced oxidative stress via the upregulation of Nrf2 expression resulting in improved intestinal barrier function.

### Example 23

### The Effects of LDNPs are Regulated by an AhR Signaling Pathway

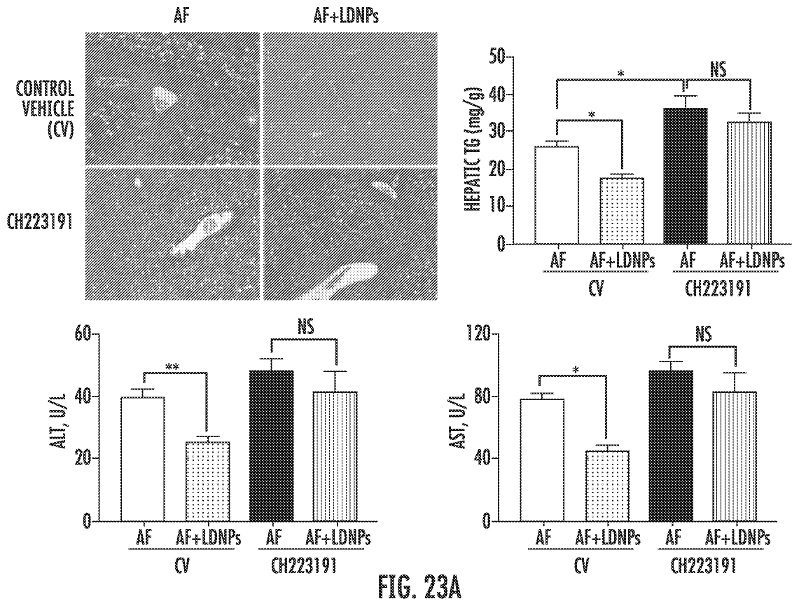

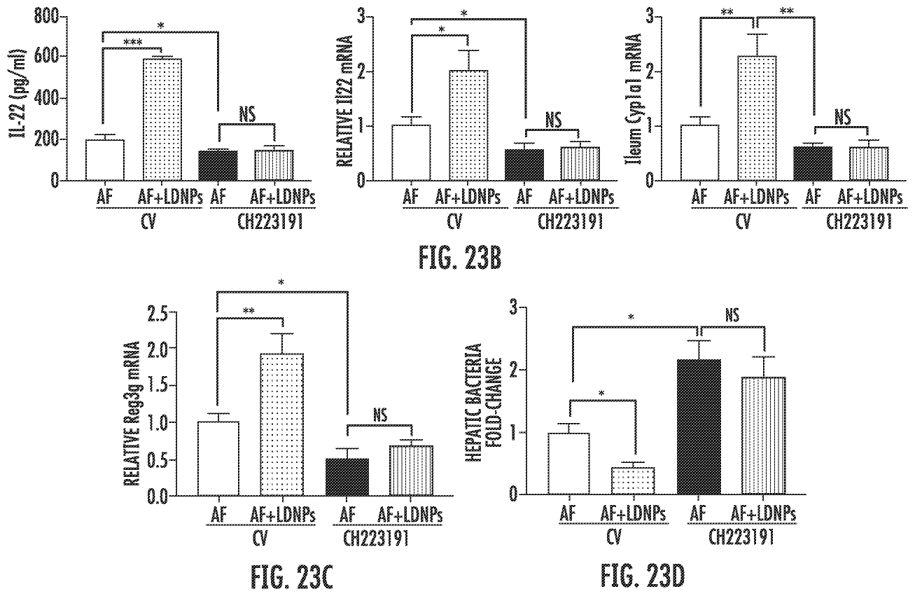

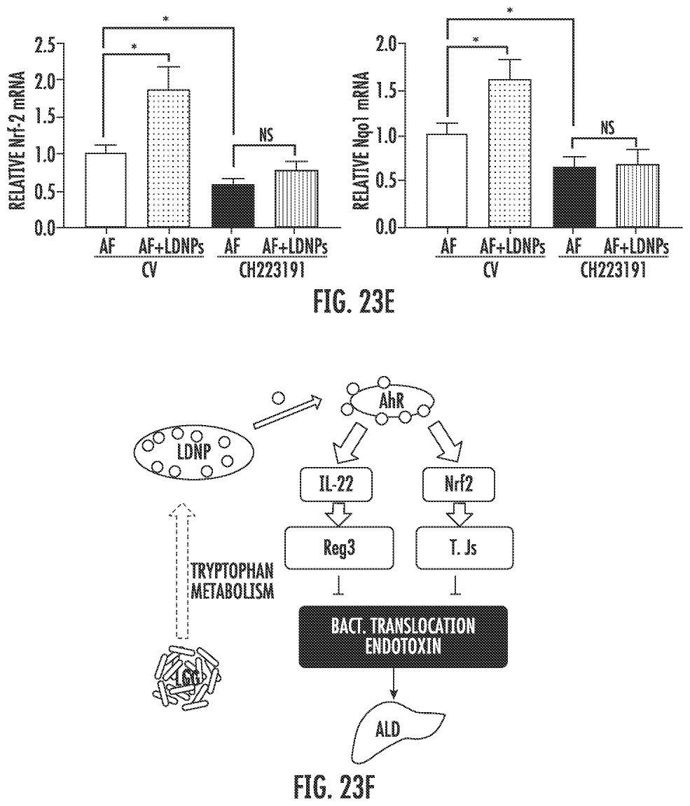

Whether the beneficial effects of LDNPs in ALD were mediated by the intestinal AhR pathway was tested by administrating the AhR inhibitor CH223191 to alcohol-fed mice. As shown in FIG. 23A, CH223191 significantly increased hepatic fat accumulation and liver triglyceride assay confirmed the histological findings. Importantly, the suppressive effect on hepatic fat by LDNP treatment was blunted when CH223191 was co-administered. CH223191 slightly increased serum levels of ALT and AST and blocked the effects of LDNPs when the mice were co-fed with CH223191.

The effects of this AhR inhibitor on downstream signaling were then determined. Ileum 1122 mRNA and protein expression was reduced by CH223191 in alcohol-fed mice. LNDPs treatment alone significantly increased IL-22 expression in alcohol-fed mice, and this effect was completely inhibited when CH223191 was co-administered (FIG. 23B, left and middle panels). Cyp1a1 mRNA expression was increased by LDNP treatment and reduced by CH223191, and no change was found when LNDPs were gavaged with the inhibitor (FIG. 23B, right panel). Similar to the IL-22 regulation, it was determined that Reg3g mRNA expression was increased by LDNPs and reduced by CH223191; and the upregulation of Reg3g by LDNPs was blocked by CH223191 (FIG. 23C). As a result, bacterial translocation, as determined by hepatic bacteria load, was significantly increased by the inhibitor and the effect of LDNPs was blocked when the inhibitor was co-administered (FIG. 23D).

Additionally, it was determined that ileum Nrf2 and Nqo1 mRNA expression was reduced by the AhR inhibitor in AF mice, and again, LDNPs were not able to increase the expression of these anti-oxidant molecules when administered together with CH223191 (FIG. 23E). These data indicated that the preventive effects of LDNPs on the alcohol-induced bacterial translocation into the liver was mediated by an AhR-regulated signaling pathway.

### Discussion of the EXAMPLES

Intestinal bacterial microbiome homeostasis is maintained under physiological conditions. Interruption of this balance is often associated with disease development and/or progression. Administration of probiotics in preclinical studies and clinical practice has shown beneficial effects in restricting the overgrowth of pathogenic bacteria and control of the pathophysiological processes in the host. As noted herein, probiotics have been used for the management of ALD to normalize the gut microbiota dysbiosis and attenuate liver injury (Kirpich et al. (2008) Probiotics restore bowel flora and improve liver enzymes in human alcohol-induced liver injury: a pilot study. Alcohol 42:675-682).

The action of bacteria on host cells is a complex process that is incompletely understood. Recent studies suggested that the exosome-like nanoparticle (ELNP) is one of the important mediators of cell-cell interaction. These kinds of nanoparticles are produced by almost all organisms including bacteria (Deatherage & Cookson (2012) Membrane Vesicle Release in Bacteria, Eukaryotes, and Archaea: a Conserved yet Underappreciated Aspect of Microbial Life. Infection and Immunity 80:1948-1957). While it is well known that Gram-negative bacteria produce ELNPs, recent studies further demonstrated that Gram-positive bacteria can also release ELNPs, despite having a thick cell wall (see e.g., Nahui Palomino et al. (2019) Extracellular vesicles from symbiotic vaginal lactobacilli inhibit HIV-1 infection of human tissues. Nat Commun 10:5656; Brown et al. (2015) Through the wall: extracellular vesicles in Gram-positive bacteria, mycobacteria and fungi. Nature Reviews Microbiology 13:620-630; Lee et al. (2009) Gram-positive bacteria produce membrane vesicles: proteomics-based characterization of Staphylococcus aureus-derived membrane vesicles. Proteomics 9:5425-5436).

LGG is one of the best-characterized Gram-positive probiotics, and as disclosed herein, LGG function was examined to determine if it was mediated through its ELNPs to regulate intestinal function in mice with experimental ALD. ALD is characterized by gut barrier dysfunction that leads to increased bacterial translocation and endotoxin release into circulation. As disclosed herein, administration of LGG viable bacteria and LGG culture supernatant (LGGs) prevented ALD in mice. Additionally and as also disclosed herein, three-day administration of LGG-derived exosome-like NPs (LDNPs) was able to reverse/prevent alcohol-induced hepatic fat accumulation, liver enzyme elevation, inflammation, and apoptotic cell death in mice using the binge-on-chronic alcohol (10d+1b) model.

LGG produces ELNPs (LDNPs) with an average size of 75 nm. The culture medium, MRS, also contains NPs, but with a bigger size and in a much smaller amount. LDNPs do not contain the eukaryotic EV marker (CD63) but have LGG-derived proteins (p75 and p40). LDNPs are disclosed herein to be pH2.2 resistant, which makes LDNPs suitable for oral administration. Indeed, it has been shown that ELNPs protect their cargo, including proteins, metabolites, and genetic material such as miRNA and mRNA from enzymatic degradation (van Niel et al. (2018) Shedding light on the cell biology of extracellular vesicles. Nat Rev Mol Cell Biol 19:213-228).

Further demonstrated herein is that the LDNPs localized mainly in the intestine tissue and immune cells when orally gavaged. This unique feature of intestinal targeting of LDNPs provides a strategy suitable for the treatment of diseases with intestinal dysfunction as an etiology.

The observation that macrophages take up LDNPs led to the examination of LDNP anti-inflammatory activity. LDNPs are shown to have reduced LPS-stimulated expression of pro-inflammatory mediators in RAW264.7 macrophages, mouse peritoneal macrophages (PMs), and bone marrow-derived macrophages (BMDMs). Analyzing the cargo composition showed that LDNPs contain abundant IA, I3A, and ILA (indol-3-lactic acid), which are tryptophan bacterial metabolites and endogenous AhR ligands. Indeed, treatment with LDNPs significantly increased AhR reporter activity and downstream AhR signaling. Importantly, the results disclosed herein demonstrated that those AhR ligands were enriched in LDNPs, since depleting LDNPs decreased AhR ligands content in LGG culture supernatant (LGGs) and blunted the AhR reporter activity of LGGs.

AhR is a ligand-activated nuclear receptor. Intestinal AhR activation in RAR-related orphan receptor gamma (RORY)-expressing ILC-3 cells leads to increased IL-22 expression and usually confers protection from bacterial infection and translocation by increasing Paneth cell-produced antimicrobial peptides, REG3Y and REG30 (28, 32). Demonstrated herein is that LDNP treatment led to an increased AhR activity as reflected by increased expression of Cyp1a1 and IL-22 in LPLs. The role of AhR in the LDNP-mediated IL-22 increase was further demonstrated by using an AhR inhibitor, which completely blocked the effects of LDNPs on IL-22 production. It was found that LDNP treatment resulted in an increase of Reg3 expression in the ileum. It is thus clear that oral LDNPs administration upregulates the intestinal AhR-IL22-Reg3 signaling pathway, which may provide protection against bacterial translocation under disease conditions.

This possibility was confirmed by the presently disclosed data in mice with experimental ALD. Three-day LDNPs oral gavage reduced alcohol-induced hepatic bacteria load, which was associated with an upregulation of Paneth cell numbers and the expression of Reg3 and IL-22, and increased AhR activity. As a result, LDNP treatment reversed/prevented ALD in mice fed alcohol.

Intestinal tight junction-mediated barrier integrity plays a key role in alcohol-induced endotoxin release. It was found LDNPs administration to intestinal epithelial Caco-2 cells led to an increased expression of tight junction proteins. Strikingly, this increase was blocked by an AhR inhibitor, indicating that AhR mediates this effect of LDNPs. It is well-known that intestinal tight junctions are damaged by disease-initiated oxidative stress, and Nrf2 is an important mediator. Disclosed herein is the determination that LDNP treatment increased Nrf2 expression, which was blocked by the AhR inhibitor. These data indicated that the effect of LDNPs on intestinal tight junction expression was mediated by AhR via the upregulation of Nrf2.

The role of the AhR-mediated effects of LDNP in ALD mice was determined using an AhR inhibitor. Co-administration of CH223191 abolished the beneficial effects of LDNPs on alcohol-induced fatty liver and liver injury, which were associated with the blockade of IL22-Reg3-mediated reduction of bacterial translocation and Nrf2-mediated LPS release.

In conclusion, it has been determined that LDNPs protected against alcohol exposure-induced fatty liver and injury through intestinal AhR-Nrf2 signaling pathways to increase antimicrobial peptide (Reg3γ and Reg3β) and tight junction protein expression leading to reduced bacterial translocation and endotoxemia (FIG. 23F). The presently disclosed results suggested that the beneficial effects of probiotics and their supernatant were likely mediated by its exosome-like NPs released from the probiotic bacteria, supporting a new strategy for the treatment of ALD and other gut barrier dysfunction-associated diseases.

Summarily, probiotics are known to modulate intestinal barrier integrity against alcohol-induced endotoxin leakiness and bacterial translocation. The presently disclosed subject matter demonstrates that Lactobacillus rhamnosus GG (LGG) and its culture supernatant protected against alcoholic liver disease (ALD) through increasing intestinal mucus and epithelial tight junction protein expression resulting in a reduction of circulation LPS level. Recent studies demonstrated the critical role of intestinal aryl hydrocarbon receptor (AhR) in the regulation of retinoic acid-related orphan receptor gamma t-positive (RORγt⁺) innate lymphoid cell (ILC3) function to produce IL-22, which modulates intestinal microbiota homeostasis and barrier function through inducing the expression of epithelial antimicrobial peptide regenerating islet-derived proteins 3γ (Reg3γ) and 3β (Reg3β).

Nanoparticles (LDNPs) were isolated from LGG cultural supernatant and examined in the modulation of ALD. C57B6 mice were subjected to the mouse chronic plus binge ethanol (EtOH) feeding ALD model (the NIAAA model; 10 day chronic plus one binge EtOH in last day), and LDNP was supplemented starting on day 7 once a day for three days. This three-day administration of LDNPs reversed alcohol-induced hepatic steatosis and injury as demonstrated by Oil Red O staining of liver sections, serum ALT and AST levels, and hepatic apoptosis. The protective effects were associated with decreased circulation LPS concentration and hepatic bacterial load, indicating an increased gut barrier function. Further analysis showed that LDNPs activated AhR luciferase activity and increased Cyp1a1 (an AhR target) enzymatic activity. LDNPs treatment increased intestinal mRNA expression of IL22, Cyp1a1, Reg3γ, and Reg3β, which play a critical role in modulating intestinal immune response to bacteria invasion and translocation. In addition, LDNPs treatment significantly increased intestinal epithelial nuclear factor erythroid 2-related factor 2 (Nrf2) protein expression, which was associated with increased protein expression of the epithelial tight junction-associated proteins ZO-1, occludin, claudin-1. Furthermore, LDNPs inhibited LPS-induced pro-inflammatory factors TNFα, and IL1β in mRNA and protein levels in Raw264.7 macrophage cells.

Taken together, the results described herein demonstrated that LDNPs treatment protected against ALD through activation of intestinal AhR-mediated upregulation of IL22 and Reg3 and through Nrf2-mediated upregulation of intestinal tight junction proteins that led to the decreased LPS release and bacterial translocation and reversal of ALD.

### REFERENCES

All references listed in the instant disclosure, including but not limited to all patents, United States and PCT International patent applications and publications thereof, scientific journal articles, and database entries (including but not limited to Uniprot, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

It will be understood that various details of the presently disclosed subject matter can be changed without departing from the scope of the presently disclosed subject matter. Furthermore, the foregoing description is for the purpose of illustration only, and not for the purpose of limitation.

1. A probiotic bacterium-derived extracellular micro- and/or nanoparticle.

2. The probiotic bacterium-derived extracellular micro- and/or nanoparticle of claim 1, wherein the probiotic bacterium is Lactobacillus rhamnosus GG (LGG).

3. The probiotic bacterium-derived extracellular micro- and/or nanoparticle of claim 1, wherein the probiotic bacterium-derived extracellular micro- and/or nanoparticle is isolated from culture supernatant in which the probiotic bacterium is growing.

4. The probiotic bacterium-derived extracellular micro- and/or nanoparticle of claim 3, wherein the probiotic bacterium-derived extracellular micro- and/or nanoparticle is purified from the culture supernatant to a purity of at least 50%, 60%, 70%, 75%, 80%, 85%, 90%, 95%, 96%, 97%, 98%, or 99% with respect to other components of the culture supernatant.

5. A method for isolating a probiotic bacterium-derived extracellular micro- and/or nanoparticle, the method comprising growing probiotic bacterium in culture, recovering some or all of the culture medium in which the probiotic bacterium is growing, and isolating the probiotic bacterium-derived extracellular micro- and/or nanoparticle from the culture medium.

6. A method for increasing probiotic LGG growth and LGG-derived extracellular micro- and/or nanoparticle, the method comprising using amino acids and/or small molecules in LGG cultural medium in which probiotic bacterium is growing faster and/or produces enhanced amount of extracellular micro- and/or nanoparticle and/or bacterium-derived AhR ligands.

7. The method of claim 5, wherein the isolating procedure comprises use of a sucrose gradient and ultracentrifugation to separate the probiotic bacterium-derived extracellular micro- and/or nanoparticle from other components of the culture medium.

8. A method for treating a liver disease or disorder, the method comprising administering to a subject in need thereof an effective amount of the probiotic bacterium-derived extracellular micro- and/or nanoparticle of claim 1 to ameliorate at least one symptom of the liver disease or disorder.

9. The method of claim 7, wherein the liver disease or disorder is selected from the group consisting of acute liver failure (ALF), alcoholic liver disease (ALD), non-alcoholic liver disease, liver steatosis, liver fibrosis, cholestatic liver disease or any combination thereof.

10. A method for increasing intestinal aryl hydrocarbon receptor (AhR) activity, Nrf2 signaling, IL-22 expression, regenerating islet-derived 3β (Reg3β) expression, regenerating islet-derived 3γ (Reg3γ) expression, or any combination thereof, the method comprising administering to a cell, tissue or organ, optionally a cell, tissue, or organ present within a subject, the probiotic bacterium-derived extracellular micro- and/or nanoparticle of claim 1 in an amount and via a route sufficient to increase intestinal aryl hydrocarbon receptor (AhR) activity, Nrf2 signaling, IL-22 expression, Reg3β expression, Reg3γ expression, or any combination thereof in the cell, tissue or organ.

11. A method for maintaining gut microbiota homeostasis, preventing or reducing bacterial intestinal transcytosis, or any combination thereof, the method comprising administering to a cell, tissue or organ, optionally a cell, tissue, or organ present within a subject, the probiotic bacterium-derived extracellular micro- and/or nanoparticle of claim 1 in an amount and via a route sufficient to maintain gut microbiota homeostasis and/or prevent and/or reduce bacterial intestinal transcytosis.

12. A method for increasing intestinal tight junctions, the method comprising administering to a cell, tissue, or organ, optionally a cell, tissue, or organ present within a subject, the probiotic bacterium-derived extracellular micro- and/or nanoparticle of claim 1 in an amount and via a route sufficient to increase intestinal tight junctions.

13. A method for decreasing circulating LPS concentration, the method comprising administering to a cell, tissue, or organ, optionally a cell, tissue, or organ present within a subject, the probiotic bacterium-derived extracellular micro- and/or nanoparticle of claim 1 in an amount and via a route sufficient to decrease circulating LPS concentration.

14. A method for protecting intestinal barrier integrity against oxidative stress, optionally oxidative stress induced by alcohol, the method comprising administering to a cell, tissue, or organ, optionally a cell, tissue, or organ present within a subject, the probiotic bacterium-derived extracellular micro- and/or nanoparticle of claim 1 in an amount and via a route sufficient to protect intestinal barrier integrity against oxidative stress.

15. A method for increasing intestinal EGF secretion, the methods comprising administrating to a cell, tissue, or organ, optionally a cell, tissue, or organ present within a subject, the probiotic bacterium-derived extracellular micro- and/or nanoparticle of claim 1 in an amount and via a route sufficient to increase intestinal EGF secretion.

16. A method for increasing HB-EGF activation, the methods comprising administrating to a cell, tissue, or organ, optionally a cell, tissue, or organ present within a subject, the probiotic bacterium-derived extracellular micro- and/or nanoparticle of claim 1 in an amount and via a route sufficient to increase macrophage HB-EGF cleavage and activation.

17. The method of claim 8, wherein the administering is associated with upregulation of intestinal Nrf2 signaling.

18. The probiotic bacterium-derived extracellular micro- and/or nanoparticle of claim 1, wherein the probiotic bacterium-derived extracellular micro- and/or nanoparticle encapsulates or is otherwise associated with a tryptophan catabolic metabolite, optionally indoleacrylic acid (IA), indole-3-aldehyde (I3A), 3-methyleneoxindole, indole, indole-3-lactic acid (ILA), indole acetic acid (IAA), or any combination thereof.


SEQUENCE LISTING (24 sequences)

SEQ ID NO: 1 - DNA, 20 bp, Artificial Sequence
  Artificially synthesized oligonucleotide primer
ccagccgatgggttgtacct

SEQ ID NO: 2 - DNA, 20 bp, Artificial Sequence
  Artificially synthesized oligonucleotide primer
tgacggcagagaggaggttg

SEQ ID NO: 3 - DNA, 23 bp, Artificial Sequence
  Artificially synthesized oligonucleotide primer
ttcatctttgaagaagagcccat

SEQ ID NO: 4 - DNA, 20 bp, Artificial Sequence
  Artificially synthesized oligonucleotide primer
tcggagcctgtagtgcagtt

SEQ ID NO: 5 - DNA, 22 bp, Artificial Sequence
  Artificially synthesized oligonucleotide primer
taccacttcacaagtcggaggc

SEQ ID NO: 6 - DNA, 23 bp, Artificial Sequence
  Artificially synthesized oligonucleotide primer
ctgcaagtgcatcatcgttgttc

SEQ ID NO: 7 - DNA, 19 bp, Artificial Sequence
  Artificially synthesized oligonucleotide primer
cagccagatgcagttaacg

SEQ ID NO: 8 - DNA, 20 bp, Artificial Sequence
  Artificially synthesized oligonucleotide primer
tctctcttgagcttggtgac

SEQ ID NO: 9 - DNA, 19 bp, Artificial Sequence
  Artificially synthesized oligonucleotide primer
ccagctactcccaggttgc

SEQ ID NO: 10 - DNA, 20 bp, Artificial Sequence
  Artificially synthesized oligonucleotide primer
ccaaacttgctccatgtcct

SEQ ID NO: 11 - DNA, 20 bp, Artificial Sequence
  Artificially synthesized oligonucleotide primer
tttgagagagtgctcgtagc

SEQ ID NO: 12 - DNA, 21 bp, Artificial Sequence
  Artificially synthesized oligonucleotide primer
ggtcttcttattctggaaagg

SEQ ID NO: 13 - DNA, 22 bp, Artificial Sequence
  Artificially synthesized oligonucleotide primer
atgcttccccgtataaccatca

SEQ ID NO: 14 - DNA, 22 bp, Artificial Sequence
  Artificially synthesized oligonucleotide primer
ggccatatctgcatcataccag

SEQ ID NO: 15 - DNA, 23 bp, Artificial Sequence
  Artificially synthesized oligonucleotide primer
actccctgaagaatataccctcc

SEQ ID NO: 16 - DNA, 22 bp, Artificial Sequence
  Artificially synthesized oligonucleotide primer
cgctattgagcacagatacgag

SEQ ID NO: 17 - DNA, 23 bp, Artificial Sequence
  Artificially synthesized oligonucleotide primer
gacccttacaagtatttggtcgt

SEQ ID NO: 18 - DNA, 21 bp, Artificial Sequence
  Artificially synthesized oligonucleotide primer
ggtatccagagccagtaacct

SEQ ID NO: 19 - DNA, 20 bp, Artificial Sequence
  Artificially synthesized oligonucleotide primer
aatcagctcagctcctgtca

SEQ ID NO: 20 - DNA, 20 bp, Artificial Sequence
  Artificially synthesized oligonucleotide primer
tcgccttgatctctccactc

SEQ ID NO: 21 - DNA, 21 bp, Artificial Sequence
  Artificially synthesized oligonucleotide primer
aggtcggtgtgaacggatttg

SEQ ID NO: 22 - DNA, 23 bp, Artificial Sequence
  Artificially synthesized oligonucleotide primer
tgtagaccatgtagttgaggtca

SEQ ID NO: 23 - DNA, 20 bp, Artificial Sequence
  Artificially synthesized oligonucleotide primer
gtaacccgttgaaccccatt

SEQ ID NO: 24 - DNA, 20 bp, Artificial Sequence
  Artificially synthesized oligonucleotide primer
ccatccaatcggtagtagcg